(19) United States
(12) Patent Application Publication
(10) Pub. No.: US 2021/0386837 A1
(43) Pub. Date: Dec. 16, 2021

(54) ACTIVE LOW MOLECULAR WEIGHT VARIANTS OF ANGIOTENSIN CONVERTING ENZYME 2 (ACE2) FOR THE TREATMENT OF DISEASES AND CONDITIONS OF THE EYE

(71) Applicant: Northwestern University, Evanston, IL (US)

(72) Inventors: Daniel Batlle, Evanston, IL (US); Jan Wysocki, Evanston, IL (US); Robert Lavker, Evanston, IL (US)

(21) Appl. No.: 17/303,032

(22) Filed: May 18, 2021

Related U.S. Application Data
(60) Provisional application No. 63/026,617, filed on May 18, 2020.

(51) Int. Cl. A61K 38/48; A61K 45/06; A61P 27/02

(52) U.S. Cl. CPC A61K 38/4813; A61P 27/02; A61K 45/06

(57) ABSTRACT

Disclosed herein are compositions and methods useful for treating diseases and conditions of the cornea in a subject in need thereof, comprising administering a therapeutically effective amount of an angiotensin converting enzyme 2 (ACE2) and/or variants of ACE2 to the subject. The disclosed variants of ACE2 may include fragments of ACE2 having ACE2 biological activity for converting AngII (1-8) to Ang (1-7).

### CROSS-REFERENCED TO RELATED PATENT APPLICATIONS

The present application claims the benefit of priority under 35 U.S.C. § 119(e) to U.S. Provisional Application No. 63/026,617, filed on May 18, 2020, the content of which is incorporated herein by reference in its entirety.

### STATEMENT REGARDING FEDERALLY SPONSORED RESEARCH OR DEVELOPMENT

This invention was made with government support under grant number DK104785 awarded by the National Institutes of Diabetes and Digestive and Kidney Disease. The government has certain rights in the invention.

### SEQUENCE LISTING

A Sequence Listing accompanies this application and is submitted as an ASCII text file of the sequence listing named “702581_01961_ST25.txt” which is 46 KB in size and was created on Jul. 1, 2021. The sequence listing is electronically submitted via EFS-Web with the application and is incorporated herein by reference in its entirety.

### BACKGROUND

Angiotensin I converting enzyme 2 (ACE2) is a critical component of the renin-angiotensin system (RAS), due to its ability to hydrolyze angiotensin II (AngII)^{1, 2, 3}. AngII is the major effector peptide of RAS and regulates cell growth, and key events in the inflammatory process⁴. In its pro-inflammatory mode, AngII directly stimulates proinflammatory mediators resulting in the infiltration of macrophages; moreover it is profibrotic and may foster angiogenesis (⁴ and references therein). The expression of ACE2 is very abundant in the kidney and intestine, followed by testis and the heart^{5, 6}. ACE2 is present in the retina⁷ but there is limited information regarding expression in the cornea. Herein we report our investigation of the role of ACE2 and Ang II in the cornea, and our development of novel therapies for corneal diseases.

### SUMMARY

Disclosed herein are compositions and methods useful for treating diseases and conditions of the cornea in a subject in need thereof, comprising administering a therapeutically effective amount of an angiotensin converting enzyme 2 (ACE2) and/or variants of ACE2 to the subject. The disclosed variants of ACE2 include fragments of ACE2 having ACE2 biological activity for converting AngII (AngII (1-8)) to Ang (1-7) and having a lower molecular weight than full-length ACE2. Preferably, the ACE2 fragments are soluble. Variants of ACE2 may further include ACE2 fragments fused to a fusion partner.

The disclosed ACE2 polypeptides and variants of ACE2 may be useful for treating disease or conditions of the cornea that include, but are not limited to, conditions in which the cornea exhibits one or more of the following: cloudiness, inflammation, deformation, expression of higher than normal levels of AngII, inflammatory markers, and/or reactive oxygen species (ROS). By way of example, but not by way of limitation, diseases or conditions of the cornea that can be treated by the methods and compositions disclosed herein include, but are not limited to, bullous keratopathy, pterygium, corneal ulcer, herpes simplex keratitis, herpes zoster ophthalmicus, herpes zoster keratitis, fungal keratitis, interstitial keratitis, keratoconjunctivitis sicca, keratomalacia, peripheral ulcerative keratitis, phlyctenular keratoconjunctivitis, superficial punctate keratitis, keratoconus, corneal dystrophies such as, but not limited to, Fuch's Dystrophy, Lattice Dystrophy Type I, Lattice Dystrophy Type II, congenital stromal corneal dystrophy, Meesmann corneal dystrophy, and map dot fingerprint dystrophy, congenital corneal diseases such as, but not limited to, complete LCAT deficiency, fish-eye disease, keratitis-ichthyosis-deafness syndrome, Peters anomaly, Peters plus syndrome; Bowen disease, Cogan syndrome, dry eye, dryness in the eye due to Sjogren syndrome, vitamin A deficiency, or LASIK eye surgery, infections, sensitivity to non-infectious bacteria or toxins, allergies, trachoma, river blindness, corneal transplant, recurrent corneal erosion, tumors, and trauma. In some embodiments, the disclosed ACE2 composition are useful to treat inflammation in the cornea of a subject in need thereof.

### BRIEF DESCRIPTION OF THE DRAWINGS

FIG. 1. Human corneal and limbal epithelial cells express AngII and ACE2. Immunohistochemical staining detects AngII (a, b) and ACE2 (c, d) in human limbal (a, c) and corneal (b, d) epithlia. N=3.

FIG. 2. ACE2-deficient corneal epithelium of young adult mice with normal appearing corneas shows increased AngII expression. Immunostaining detects AngII (a, b) and ACE2 (c, d) in WT (a, c) and Ace2^{-/-} (b, d) epithelia. N=3.

FIG. 3. Aged ACE2-deficient mice show corneal inflammation and epithelial defect. (a-d) Representive clinical images of WT (a) and Ace2^{-/-} mouse eyes (b-d) were taken using a dissecting scope. (e-h) Representive H&E stained histological images of WT (e) and Ace2^{-/-} (f-h) mouse corneas. N=6.

FIG. 4. Aged ACE2-deficient mice show infiltration of immune cells. WT (a, c, e) and Ace2^{-/-} (b, d, f) mouse corneas were stained for CD68 (marker for macrophage), CD3 (marker for T cells), and CD11c (marker for dendritic cells). N=3.

FIG. 5. Young adult ACE2-deficient mice show corneal haze after central corneal debridement wounding. (a) Clinical and fluorescein images were taken at 1, 3 and 7 days post injury. Green fluorescence represents areas devoid of epithelium (i.e., corneal wounds). Corneal haze was observed in ACE2^{-/-} mouse corneas at day 7 post injury. N=6. (b) Bar graph showing Clinical Haze scores derived on day 0, day 1, day 3 and day 7.

FIG. 6. Young adult ACE2-deficient mice show corneal inflammation and epithelial defect after central corneal debridement wounding. WT (a, b, e, f) and Ace2^{-/-} (c, d, g, h) mouse corneas were stained with H&E. Representative histological images of WT (a, b, e, f) and Ace2^{-/-} (c, d, g, h) mouse corneas show a dramatic infiltration of immune cells in Ace2^{-/-} but not in WT mouse corneas at day 1 post injury (a-d) and at day 7 post injury (e-h). Each image is representative of an individual mouse. N=8.

FIG. 7. Losartan treatment can partially reverse pro-inflammatory activity in ACE2 depleted corneal epithelial cells. hTCEpi cells were transfected with siControl or siACE2. siACE cells were treated with losartan for 24 hours and then compared with siControl and untreated siACE transfected cells. Total RNAs were isolated from these cells for RT-qPCR for inflammation-related genes. N=8. *p<0.05. The first bar in each grouping represents siControl transfected cells; the second bar in each grouping represents untreated siACE2 transfected cells; the third bar in each grouping represents siACE2 transfected cells treated with 100 μM losartan.

FIG. 8 provides (a) a diagram of a trans-well chemotaxis assay; (b) 20× images of macrophage cells migration in response to control hTCEpi cells, versus macrophage migration in response to hTCEpi cells treated with siACE2; (c) bar graph showing area of migrating macrophage cells.

FIG. 9. Provides bar graphs showing the change in expression of IL1α, IL1β, IL6, Tnfα, Cxcl8, and Ccl2 from macrophages exposed to media from (1) hTCEpi control cells or (2) siACE2 treated hTCEpi cells.

FIG. 10. Shows (a) the amino acid and (b) the nucleic acid sequence of human ACE2, amino acids 1-618 as SEQ ID NO:10 and SEQ ID NO:17, respectively.

FIG. 11. Shows (a) the amino acid and (b) the nucleic acid sequence of human ACE2 amino acids 1-618 fused to albumin binding domain (ABDCon) via a GS4 linker as SEQ ID NO:18 and SEQ ID NO:19, respectively.

FIG. 12. Recovery of ACE2 activity in eyes isolated from ACE2 deficient mice. Recovery of ACE2 activity in eyes isolated from ACE2 deficient mice 1 hour after topical corneal application of human recombinant (hr) ACE2 1-618ABD. Eyes of untreated ACE2 knockout mice (circles) were compared to eyes of ACE2 knockout mice to which hr ACE2 1-618ABD (5 μg/μl) was applied either dissolved in PBS (squares) or in 0.5% DMSO in PBS (triangles).

FIG. 13. Appearance of ACE2 staining by immunofluorescence in the cornea of ACE2 deficient mice after administration of ACE2 1-618ABD topically.

### DEFINITIONS

The present invention is described herein using several definitions, as set forth below and throughout the application.

A: As used in this specification and the claims, the singular forms “a,” “an,” and “the” include plural forms unless the context clearly dictates otherwise. For example, the term “a polypeptide fragment” should be interpreted to mean “one or more polypeptide fragments” unless the context clearly dictates otherwise. As used herein, the term “plurality” means “two or more.”

About: As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

Agent: In general, the term “agent”, as used herein, is used to refer to an entity (e.g., for example, a lipid, metal, nucleic acid, polypeptide, polysaccharide, small molecule, etc, or complex, combination, mixture or system [e.g., cell, tissue, organism] thereof), or phenomenon (e.g., heat, electric current or field, magnetic force or field, etc). In some instances, as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form.

Amino acid sequence: The term “amino acid sequence” refers to an oligopeptide, peptide, polypeptide, or protein sequence (which terms may be used interchangeably), or a fragment of any of these, whether naturally occurring or synthetic. Where “amino acid sequence” refers to a sequence found in a naturally occurring protein molecule, “amino acid sequence” and like terms are not meant to limit the amino acid sequence to the complete native amino acid sequence associated with the recited protein molecule. In some embodiments, amino acid sequences contemplated herein may include one or more amino acid substitutions relative to a reference amino acid sequence. For example, a variant polypeptide may include non-conservative and/or conservative amino acid substitutions relative to a reference polypeptide. “Conservative amino acid substitutions” are those substitutions that are predicted to interfere least with the properties of the reference polypeptide. In other words, conservative amino acid substitutions substantially conserve the structure and the function of the reference protein. The following Table provides a list of exemplary conservative amino acid substitutions.

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally do not maintain one or more of: (a) the structure of the polypeptide backbone in the area of the substitution, for example, as a beta sheet or alpha helical conformation, (b) the charge or hydrophobicity of the molecule at the site of the substitution, and/or (c) the bulk of the side chain. In some embodiments, disclosed peptides may include an N-terminal esterification (e.g., a phosphoester modification) or a pegylation modification, for example, to enhance plasma stability (e.g. resistance to exopeptidases) and/or to reduce immunogenicity.

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the term “corresponding to” is understood to refer to a relationship between two or more entities. For example, the term “corresponding to” may be used to designate the position/identity of a structural element in a compound or composition relative to another compound or composition (e.g., to an appropriate reference compound or composition). For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190th amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure. Those of skill in the art will also appreciate that, in some instances, the term “corresponding to” may be used to describe an event or entity that shares a relevant similarity with another event or entity (e.g., an appropriate reference event or entity). To give but one example, a gene or protein in one organism may be described as “corresponding to” a gene or protein from another organism in order to indicate, in some embodiments, that it plays an analogous role or performs an analogous function and/or that it shows a particular degree of sequence identity or homology, or shares a particular characteristic sequence element.

Deletion: A “deletion” refers to a change in a reference amino acid sequence (e.g., SEQ ID NO:1 or SEQ ID NO:2) that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant” of a reference polypeptide sequence may include a deletion relative to the reference polypeptide sequence. For example, SEQ ID NO:3 (amino acids 1-619), SEQ ID NO:4 (amino acids 1-605), SEQ ID NO:10 (amino acids 1-618), SEQ ID NO:11 (amino acids 1-542), and SEQ ID NO:12 (amino acids 1-522) include C-terminal deletions relative to reference sequence SEQ ID NO:1 (amino acids 1-805).

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been subjected to a manipulation, so that its genetic, epigenetic, and/or phenotypic identity is altered relative to an appropriate reference cell such as otherwise identical cell that has not been so manipulated In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

Fragment: A “fragment” is a portion of an amino acid sequence which is identical in sequence to but shorter in length than a reference sequence (e.g., SEQ ID NO:1 or SEQ ID NO:2). A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide; or a fragment may comprise no more than 5, 10, 15, 20, 25, 30, 40, 50, 60, 70, 80, 90, 100, 150, 250, or 500 contiguous amino acid residues of a reference polypeptide; or a fragment may comprise a range of contiguous amino acid residues of a reference polypeptide bounded by any of these values (e.g., 40-80 contiguous amino acid residues). Fragments may be preferentially selected from certain regions of a molecule. The term “at least a fragment” encompasses the full length polypeptide. A “variant” of a reference polypeptide sequence may include a fragment of the reference polypeptide sequence. For example, SEQ ID NO:3 (amino acids 1-619), SEQ ID NO:4 (amino acids 1-605), SEQ ID NO:10 (amino acids 1-618), SEQ ID NO:11 (amino acids 1-542), and SEQ ID NO:12 (amino acids 1-522) comprise fragments of reference sequence SEQ ID NO:1 (amino acids 1-805).

Fusion polypeptide: A “fusion polypeptide” refers to a polypeptide comprising at the N-terminus, the C-terminus, or at both termini of its amino acid sequence a heterologous (i.e., exogenous) amino acid sequence, for example, a heterologous amino acid sequence that extends the duration of action of the fusion polypeptide extends the duration of action of the ACE2 variant in plasma and organs, such as lungs, of a subject who has been administered the fusion polypeptide. In some embodiments, the heterologous or exogenous amino acid sequence extends the duration of action of the fusion polypeptide by increasing the half-life of the fusion polypeptide in plasma and organs, such as lungs, of a subject who has been administered the fusion polypeptide. A “variant” of a reference polypeptide sequence may include a fusion polypeptide comprising the reference polypeptide fused to a heterologous or exogenous sequence.

Homology: “Homology” refers to sequence similarity or, interchangeably, sequence identity, between two or more polypeptide sequences. Homology, sequence similarity, and percentage sequence identity may be determined using methods in the art and described herein.

Identity: The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

Include: As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

Insertion: The words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, or 200 amino acid residues or a range of amino acid residues bounded by any of these values (e.g., an insertion or addition of 5-10 amino acids). A “variant” of a reference polypeptide sequence may include an insertion or addition relative to the reference polypeptide sequence.

Polypeptide: As used herein refers to a polymeric chain of amino acid moieties linked together by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, glycosylation, lipidation, methylation, pegylation, phosphorylation etc., including combinations thereof. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Specific binding: As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Subject: As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like. The disclosed methods, compositions, and kits may be utilized to treat a subject in need thereof. A “subject in need thereof” is intended to include a subject having or at risk for developing diseases and disorders of the eye, including diseases and disorders of the cornea.

Variant: In some embodiments, a “variant” of a particular polypeptide sequence may be defined as a polypeptide sequence having at least 20% sequence identity to the particular polypeptide sequence over a certain length of one of the polypeptide sequences using blast with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides, or range of percentage identity bounded by any of these values (e.g., range of percentage identity of 80-99%).

### DETAILED DESCRIPTION

Angiotensin converting enzyme 2 (ACE2) plays an important role in inflammation, which is attributable at least, in part, to the conversion of the pro-inflammatory angiotensin (Ang II) peptide into angiotensin 1-7 (Ang 1-7), a peptide which opposes the actions of AngII. AngII comprises 8 amino acids, and is also known as AngII (1-8). ACE2 and AngII are present in many tissues but information on the cornea is lacking.

During our investigations using ACE2-deficient mice, we noted that as the ACE2-deficient mice aged, some developed cloudy corneas. In certain mice, cloudy corneas were bilateral, in others they were unilateral, whereas some adult aged mice had clear corneas. Herein, we report that AngII and ACE2 are expressed in limbal and corneal epithelia in humans and mice. Moreover, when challenged with corneal injury, ACE2-deficient mice are “primed” for an increased corneal inflammatory response. Once initiated, inflammation persists, which markedly alters the epithelial and stromal phenotypes. Blockade of the AngII type 1 receptor (AT1R) partially restores the cytokine/chemokine imbalance due to ACE2 deficiency. Collectively, our findings establish a pivotal role of ACE2 in the cornea and identifies AngII blockade as a potential new target for corneal inflammation. Moreover, the administration of ACE2 protein is anticipated to have a therapeutic benefit by fostering the degradation of AngII and the formation of Ang 1-7 as has been proposed for other disease entities⁸.

The disclosed methods and compositions may be utilized to treat a subject in need thereof. A “subject in need thereof” is intended to include a subject having or at risk for developing diseases or disorders of the cornea. In addition to trauma, exemplary disease or conditions that negatively affect the cornea and that may be treated via the compositions and methods herein, include disease or conditions exhibiting one or more of corneal deformation, corneal clouding, corneal inflammation, corneal neovascularization, corneal edema, and/or the increased expression of one or more markers of inflammation, AngII, and ROS. Exemplary diseases and conditions include, but are not limited to bullous keratopathy, pterygium, corneal ulcer, herpes simplex keratitis, herpes zoster ophthalmicus, herpes zoster keratitis, fungal keratitis, interstitial keratitis, keratoconjunctivitis sicca, keratomalacia, peripheral ulcerative keratitis, phlyctenular keratoconjunctivitis, superficial punctate keratitis, keratoconus, corneal dystrophies such as, but not limited to Fuch's Dystrophy, Lattice Dystrophy Type I, Lattice Dystrophy Type II, congenital stromal corneal dystrophy, Meesmann corneal dystrophy, and map dot fingerprint dystrophy, congenital corneal diseases such as, but not limited to complete LCAT deficiency, fish-eye disease, keratitis-ichthyosis-deafness syndrome, Peters anomaly, Peters plus syndrome; Bowen disease, Cogan syndrome, dry eye, dryness in the eye due to Sjogren syndrome, vitamin A deficiency, or LASIK eye surgery, infections, sensitivity to non-infectious bacteria or toxins, allergies, trachoma, river blindness, corneal transplant, recurrent corneal erosion, and tumors. In some embodiments, the disclosed methods and composition are useful to treat inflammation in the cornea of a subject in need thereof.

ACE2

The disclosed methods of treatment and pharmaceutical composition utilize and/or include angiotensin converting enzyme 2 (ACE2) or variants thereof such as fragments of ACE2 and fusions thereof. The nucleotide sequence of the human ACE2 gene is available from the National Center for Biotechnology Information of the National Institutes of Health. The location of the human ACE2 gene is provided as NC_000023.11 (15494525 . . . 15602069, complement). ACE2, isoform 1, is a transmembrane protein which is expressed first as a precursor polypeptide having the amino acid sequence (SEQ ID NO:1). The mouse (Mus musculus) homolog of ACE2 has the following amino acid sequence (SEQ ID NO:2).

ACE2 is naturally produced with a leader polypeptide, that is cleaved in production of mature protein. Reports have described amino acids 1-17 of the precursor ACE2 polypeptide as corresponding to the leader peptide which is cleaved from mature ACE2. Amino acids 18-740 are extracellular. Amino acids 741-761 form a helical transmembrane sequence. Amino acids 762-805 are cytoplasmic. Those skilled in the art will appreciate that the exact number of amino acids in any of these portions (e.g., leader peptide, extracellular, transmembrane, cytoplasmic) may vary from these, and will readily be able to determine appropriate corresponding residues, for example using standard sequence comparison and/or assessment technologies.

ACE2 exhibits molecular functions that may include: carboxypeptidase activity, endopeptidase activity, glycoprotein binding activity, metallocarboxypeptidase activity, and zinc ion binding activity. In some embodiment, the variants of ACE2 disclosed herein, including fragments of ACE2 and fusion thereof, function to at least cleave Angiotensin II (e.g., function to convert AngII to Ang (1-7)). In some embodiments, variants include all of the molecular and enzymatic functions of ACE2.

In some embodiments, the variants of ACE2 include or lack one or more of the following features: amino acid position 169—chloride binding site; amino acid position 273—substrate binding site; amino acid position 345 substrate binding site; amino acid position 346—substrate binding site via a carbonyl oxygen; amino acid position 371—substrate binding site; amino acid position 374—metal binding site (e.g., Zn²⁺); amino acid position 375—active site; amino acid position 378—catalytic metal binding site (e.g. Zn²⁺); amino acid position 402—catalytic metal binding site (e.g. Zn²⁺); amino acid position 477—chloride binding site; amino acid position 481—chloride binding site; amino acid position 505—active site; and amino acid position 515 substrate binding site. In some embodiments, variants of ACE2 disclosed herein, including fragments of ACE2, include one or more of soluble human recombinant ACE2 1-618, and/or human ACE2 1-618ABD protein which contain all N-terminal 618 amino acid residues. Human ACE2 1-618 ABD containing albumin binding domain has an extended duration of action in vivo and in some embodiments, has advantages for practical use. In some embodiments, for instance, one injection lasts for at least 4 days as determined by enzymatic activity in plasma and by biologic response (blood pressure). In addition, the short variants do not contain a ferredoxin-like fold domain, referred to as the Neck domain (residues 616-726) which is important for dimerization of ACE2 protein. Accordingly, these variants are not expected to form dimers as the full-length ACE2 does. Thus not only by virtue of amino acid sequence but also by the expected lack of dimerization, these variants have a smaller molecular size than the full length ACE2, and thus could be suitable for delivery, particularly for delivery to the eye, and particularly the cornea.

In some embodiments, the variants of ACE2 include or lack one or more of the following features: amino acid positions 23-52—helix; amino acid positions 56-77; amino acid positions 78-82—turn; amino acid positions 85-87—helix; amino acid positions 91-100—helix; amino acid positions 104-107—helix; amino acid positions 110-129—helix; amino acid positions 131-134—beta strand; amino acid positions 137-143—beta strand; amino acid positions 144-146—turn; amino acid positions 148-154—helix; amino acid positions 158-171—helix; amino acid positions 173-193—helix; amino acid positions 196-198—beta strand; amino acid positions 199-204—helix; amino acid positions 205-207—turn; amino acid positions 213-215—turn; amino acid positions 220-251—helix; amino acid positions 253-255—turn; amino acid positions 258-260-beta strand; amino acid positions 264-266—helix; amino acid positions 267-271—beta strand; amino acid positions 279-282—helix; amino acid positions 284-287—turn; amino acid positions 294-297—turn; amino acid positions 298-300—helix; amino acid positions 304-316—helix; amino acid positions 317-319—turn; amino acid positions 327-330—helix; amino acid positions 338-340—beta strand; amino acid positions 347-352—beta strand; amino acid positions 355-359—beta strand; amino acid positions 366-384—helix; amino acid positions 385-387—turn; amino acid positions 390-392—helix; amino acid positions 400-413—helix; amino acid positions 415-420-helix; amino acid positions 422-426—turn; amino acid positions 432-446—helix; amino acid positions 449-465—helix; amino acid positions 466-468—beta strand; amino acid positions 473-483—helix; amino acid positions 486-488—beta strand; amino acid positions 499-502—helix; amino acid positions 504-507—helix; amino acid positions 514-531—helix; amino acid positions 532-534—turn; amino acid positions 539-541—helix; amino acid positions 548-558—helix; amino acid positions 559-562—turn; amino acid positions 566-574—helix; amino acid positions 575-578—beta strand; amino acid positions 582-598—helix; amino acid positions 600-602—beta strand; and amino acid positions 607-609—beta strand. In some embodiments, variants of ACE2 disclosed herein, including fragments of ACE2, include one or more of an addition of albumin binding domain (ABDCon) linked to ACE2 by, for example, a GS4 linker (see e.g., SEQ ID NO: 18).

Natural variants of ACE2 are contemplated herein and may include the natural variant K26R and the natural variant N638S. Natural isoforms of ACE2 also are contemplated herein include isoform 2 and the differences of its amino acid sequence relative to the amino acid sequence of isoform 1: F555L and/or Δ556-805. Variants of ACE2 disclosed herein, including fragments of ACE2, may have or lack one or more of these amino acid sequences of ACE2.

In some embodiments, the variants of ACE2 are soluble in an aqueous solution. For example, variants of ACE2 may include variants that include a deletion which removes the transmembrane domain of ACE2 and/or the cytoplasmic portion of ACE2.

In some embodiments, the variants of ACE2 include one or more of the following amino acid modifications: amino acid position 53—N-linked glycosylation; amino acid position 90—N-linked glycosylation; amino acid position 103—N-linked glycosylation; amino acid positions 133←→141—disulfide bond; amino acid position 322—N-linked glycosylation; amino acid positions 344←→361—disulfide bond; amino acid position 432—N-linked glycosylation; amino acid positions 530←→542; amino acid position 546—N-linked glycosylation; and amino acid position 690—N-linked glycosylation.

ACE2 regulates biological processes that include: angiotensin catabolism processes in blood, angiotensin maturation processes, angiotensin-mediated drinking behavior processes, positive regulation of cardiac muscle contraction processes, positive regulation of gap junction assembly processes, positive regulation of reactive oxygen species metabolism processes, receptor biosynthesis processes, receptor-mediated virion attachment processes (e.g., coronaviruses), regulation of cardiac conduction processes, regulation of cell proliferation processes, regulation of cytokine production processes, regulation of inflammatory response processes, regulation of systemic arterial blood pressure by renin-angiotensin processes, regulation of vasoconstriction processes, regulation of vasodilation processes, tryptophan transport processes, and viral entry into host cell processes (e.g., coronaviruses). In some embodiments, variants of ACE2 disclosed herein, including fragments of ACE2, function to regulate one or more of these biological processes.

Provided ACE2 Variants

The present disclosure provides and/or utilizes certain variants of ACE2, and particularly of human ACE2. Provided variants are preferably soluble.

In some embodiments, the present disclosure provides and/or utilizes ACE2 variants that provide (e.g., retain) one or more ACE2 protein activities (e.g., molecular and/or enzymatic activities). For example, in many embodiments, provided soluble human ACE2 variant polypeptide agents are characterized by molecular functions and/or enzymatic functions that may include, for example: carboxypeptidase activity, endopeptidase activity, glycoprotein binding activity, metallocarboxypeptidase activity, cleavage of Angiotensin II, and zinc ion binding activity.

Among other things, the present disclosure provides an insight that ACE2 variants characterized both by binding and enzymatic activity are particularly useful and/or provide one or more unusual or surprising benefits for treatment of eye diseases and disorders, including diseases and disorders of the cornea.

In some embodiments, soluble ACE2 variant polypeptide agents for use in accordance with the present disclosure are or comprise a fragment (e.g., a truncation) of ACE2; alternatively or additionally, in some embodiments, provided ACE2 variants are or comprise a fusion or conjugate (e.g., a multimer) that includes an ACE2 moiety, which may be or comprise an ACE2 fragment (e.g., truncation).

In some embodiments, soluble ACE2 variant polypeptide agents for use in accordance with the present disclosure may have a maximum molecular weight or size. For example, in some embodiments, such agents may be less than about 250 kD, 225 kD, 200 kD, 175 kD, 150 kD, 125 kD, 100 kD, 95 kd, 90 kD, 85 kD, 80 kD, 75 kD, 70 kD, 65 kD, 60 kD, 55 kD, 50 kD, 45 kD, 40 kD, or 35 kD (or have a molecular weight of a range bounded by any of these values). Alternatively or additionally, in some embodiments, a provided agent may be or comprise an ACE2 fragment that is 70 kD, 65 kD, 60 kD, 55 kD, 50 kD, 45 kD, 40 kD, 35 kD, 30 kD, 25 kD, 20 kD, 15 kD, 10 kD, 5 kD or less (or have a molecular weight of a range bounded by any of these molecular weight values). Active low molecular weight variants of ACE2 are described in U.S. Publication No. 2018/0230447, the content of which is incorporated herein by reference in its entirety.

In some embodiments, soluble ACE2 variant polypeptide agents may be utilized in a non-glycosylated form. In some embodiments, glycosylated forms of disclosed ACE2 variants may exhibit a higher molecular weight than the non-glycosylated forms.

In some embodiments, ACE2 variants (e.g., soluble ACE2 variant polypeptide agents) disclosed herein, whether the ACE2 variants are glycosylated or not, may have a molecular weight of a range bounded by any of the foregoing values (e.g., 250 kD, 225 kD, 200 kD, 175 kD, 150 kD, 125 kD, 100 kD, 95 kd, 90 kD, 85 kD, 80 kD, 75 kD, 70 kD, 65 kD, 60 kD, 55 kD, 50 kD, 45 kD, 40 kD, or 35 kD, and/or 70 kD, 65 kD, 60 kD, 55 kD, 50 kD, 45 kD, 40 kD, 35 kD, 30 kD, 25 kD, 20 kD, 15 kD, 10 kD, 5 kD or less).

In some embodiments, an ACE2 fragment or moiety, and particularly an ACE2 moiety included in a conjugate as described herein, may contain one or more cysteine modifications. To give but a few examples, in some embodiments, cysteines 261 and/or 498 may be replaced by other amino acids, and/or tryptophan (610) and/or alanine (614) may be replaced by cysteine. Cysteine substitutions are not limited to the above listed amino acid positions.

In some embodiments, an ACE2 fragment or moiety, and particularly an ACE2 moiety included in a conjugate as described herein, may contain only a single cysteine residue. Single-cysteine moieties are particularly useful where conjugation is via a Cys SH, among other things to improve conjugate consistency and/or homogeneity. In some such embodiments, one or more cysteines that is naturally present in the ACE2 (e.g., hACE2) amino acid sequence may be substituted or removed; alternatively or additionally, cysteine may be introduced at one or more positions which do not contain cysteine in wild type ACE2 (e.g., hACE2), for example by insertion or substitution.

Fragments/Deletions

In some embodiments, ACE2 variants provided by and/or utilized in accordance with the present disclosure may be or include fragments (e.g., deletions or truncations) of ACE2, and particularly of human ACE2.

In some embodiments, ACE2 truncations for use in accordance with the present disclosure are smaller than ACE2(1-740); in some embodiments, useful ACE2 truncations are sufficiently small to be amenable to glomerular filtration; among other things, the present disclosure appreciates that soluble ACE2 variant polypeptide agents, as described herein, that are sufficiently small to be amenable to glomerular filtration, such that they can be filtered by the glomerular filtration barrier of the kidneys for better delivery, may provide particular advantages in treatment of diseases and disorders, especially in certain patient populations (e.g., those who are suffering from or susceptible to acute kidney injury or glomerular damage).

In some embodiments, ACE2 truncations for use in accordance with the present disclosure may correspond to a fragment of ACE2 that comprises fewer than about 750 amino acids of ACE2, more preferably fewer than about 700, 690, 680, 670, 650, 640, 630, 620, 610 or 600 amino acids of ACE2.

In some embodiments, ACE2 truncations for use in accordance with the present disclosure may be described and/or initially produced or provided as including sequences that represent or correspond to leader sequences as described herein; those of ordinary skill in the art will readily appreciate, however, that in some embodiments, leader sequences are or can be removed. Thus, for example, where use of an “hACE2(1-618)” fragment is referenced, those skilled in the art will appreciate that, in some embodiments, a corresponding truncation lacking the leader sequence (e.g., missing approximately the first 15-20 amino acids, and in some embodiments the first 17, 18, or 19 amino acids) may be utilized.

In some embodiments, ACE2 truncations for use in accordance with the present disclosure may correspond to a 1-618 fragment of hACE2.

In some embodiments, polypeptide fragments of ACE2 for use in accordance with the present disclosure may include a deletion relative to full-length ACE2 (SEQ ID NO:1). In some embodiments, disclosed polypeptide fragments may include a deletion selected from an N-terminal deletion, a C-terminal deletion, and both, relative to full-length ACE2 (SEQ ID NO:1). In some embodiments the disclosed polypeptide fragments may include an internal deletion.

In some embodiments, a deletion may remove at least about 1, 2, 3, 4, 5, 10, 15, 20, 25, 50, 100, 150, 200 amino acids or more of full-length ACE2.

In some embodiments, a deletion removes one or more glycosylation sites, and as such, the polypeptide fragments of ACE2 may be less glycosylated than full-length ACE2, further reducing the molecular weight of the polypeptide fragments of ACE2 relative to full-length ACE2.

In some embodiments, disclosed polypeptide fragments of ACE2 may include one or more amino acid substitutions that removes a glycosylation site. In some embodiments, it may be desirable to utilize a variant of ACE2 that exhibits reduced glycosylation. Full-length, wild-type ACE2 has a molecular weight of approximately 110 kD and is a membrane protein (i.e., non-soluble).

Polypeptide fragments of ACE2 disclosed herein preferably are soluble, for example, wherein the polypeptide fragments include a C-terminal truncation related to full-length, wild-type ACE2 that removes a portion that includes the transmembrane region and optionally removes the C-terminal region.

As noted above, ACE2 is known to comprise 3 disulfide bonds C133-C141, C344-C361, C530-0542. There are also non-disulfide Cysteines at C261 and C498. Because truncates 1-605, 1-619 and 1-618 are enzymatically active and 1-522 is not active and because the most distal residue seen to contact the active site is Y515, the third disulfide may be essential to enzyme activity.

Fusions and Conjugates

In some embodiments, ACE2 variants provided by and/or utilized in accordance with the present disclosure may be or include constructs in which a soluble ACE2 polypeptide (i.e., a soluble portion of an ACE2 protein) is conjugated to (i.e., covalently linked to) a stabilizing entity such that duration of action of the soluble ACE2 polypeptide is extended relative to that of the non-conjugated, “naked” parent polypeptide.

In some embodiments, a stabilizing entity may be or comprise a polypeptide, so that a provided soluble ACE2 variant polypeptide is a fusion polypeptide. Alternatively or additionally, in some embodiments, a stabilizing entity may be or comprise a non-polypeptide agent, which may in some embodiments may be a polymer (e.g., a polyethylene glycol “PEG”) or comprises a polymeric portion.

In some embodiments, ACE2 polypeptide and stabilizing entity moieties are directly associated with one another; in other embodiments they may be associated via a linker.

Fusion polypeptides of ACE2 or variants thereof are disclosed herein. In some embodiments, fusion polypeptide of ACE2 or a variant thereof may include the amino acid sequence of ACE2 or a variant thereof (e.g., the amino acid sequence of a fragment of ACE2) fused to a heterologous or exogenous amino acid sequence. Preferably, the heterologous (e.g., exogenous) amino acid sequence increases duration of action of the fusion polypeptide in plasma of a subject to which such fusion polypeptide has been administered; in some embodiments, the heterologous (e.g., exogenous amino acid sequence increases half-life of the fusion polypeptide in plasma of such a subject.

Disclosed fusion polypeptides may comprise the amino acid sequence of ACE2 or a variant thereof (e.g., the amino acid sequence of a fragment of ACE2) fused directly to a heterologous (e.g., exogenous) amino acid sequence or fused via a linker sequence. In some embodiments, suitable linker sequences may include amino acid sequences of 5, 6, 7, 8, 9, 10, 11, 12, 13, 14, or 15 amino acids or more, or a range bounded by any of these values (e.g., a linker of 5-15 amino acids). In some embodiments, a linker sequence comprises only glycine residues, serine residues, and/or alanine residues.

Fusion polypeptides disclosed herein include the amino acid sequence of ACE2 or a variant thereof fused to the amino acid sequence of a heterologous (e.g., exogenous) polypeptide.

Fusion polypeptides of ACE2 or variants thereof are disclosed herein. The fusion polypeptide of ACE2 or a variant thereof may include the amino acid sequence of ACE2 or a variant thereof (e.g., the amino acid sequence of a fragment of ACE2) fused to a heterologous amino acid sequence (e.g., a fusion partner). Preferably, the heterologous amino acid sequence increases the half-life of the fusion polypeptide in plasma.

The disclosed fusion polypeptides may comprise the amino acid sequence of ACE2 or a variant thereof (e.g., the amino acid sequence of a fragment of ACE2) fused directly to a heterologous amino acid sequence or fused via a linker sequence. Suitable linker sequences may include amino acid sequences of 5, 6, 7, 8, 9, 10, 11, 12, 13, 14, or 15 amino acids or more, or a range bounded by any of these values (e.g., a linker of 5-15 amino acids) such as the amino acid sequence of SEQ ID NO:14. In some embodiments, the linker sequence comprises only glycine and serine residues.

Fusion polypeptides disclosed herein include the amino acid sequence of ACE2 or a variant thereof fused to the amino acid sequence of an antibody or to one or more fragments of an antibody, for example, the Fc portion of an antibody (constant fragment of human IgG) which preferably is devoid of its hinge region to prevent dimerization of the fusion polypeptide (e.g., SEQ ID NO:6). Fusion of short ACE2 with Fc (e.g., SEQ ID NO:6) or the monomeric CH3 Fc derivate (e.g., SEQ ID NO:7 or SEQ ID NO:8) can enable its delivery through a functional FcRn-dependent transport pathway. In other embodiments, the fusion proteins comprises the hinge region (SEQ ID NO:15) or a modified hinge region (SEQ ID NO:16). Fusion polypeptides disclosed herein include also include the amino acid sequence of ACE2 or a variant thereof fused to serum albumin or a fragment thereof, for example domain III of human serum albumin or a fragment thereof (e.g., SEQ ID NO:9). In some embodiments, the exogenous polypeptide is or comprises the ABD of streptococcal protein G or a fragment thereof, such as the C-terminal albumin binding domain 3 (ABD3) of streptococcal protein G (e.g., ABD3 from strain G148, or the ABD035 derivative (SEQ ID NO:5) or ABDCon (SEQ ID NO:13). (See, e.g., Nilvebrant et al., Comput. Struct. Biotechnol. J. 6(7): 108, March 2013 and Jacobs et al., Protein Engineering, Design and Selection, 28(10):385-393, October 2015; the contents each of which are incorporated herein by reference in their entireties). Fusion polypeptides as disclosed herein include the amino acid sequence of ACE2 or a variant thereof fused to the albumin binding domain, (ABDcon), see e.g., SEQ ID NO: 18.

Fusion polypeptide disclosed herein may include an amino acid tag sequence, for example, which may be utilized for purifying and or identifying the fusion polypeptide. Suitable amino acid tag sequences may include, but are not limited to, histidine tag sequences comprising 5-10 histidine residues.

In some embodiments, fusion polypeptides disclosed herein may include an amino acid tag sequence, for example, which may be utilized for purifying and or identifying the fusion polypeptide. Suitable amino acid tag sequences may include, but are not limited to, histidine tag sequences comprising 5-10 histidine residues. In some embodiments, such a tag sequence may itself provide or contribute to extension of duration of action (e.g., at least in part by extending half-life). Thus, in some embodiments, a heterologous polypeptide may be or comprise such tag. Alternatively, in some embodiments, a tag may not itself provide or contribute to extension of duration of action. In some embodiments, a fusion polypeptide provided by the present disclosure may comprise each of (i) an ACE2 moiety; (ii) a stabilizing moiety; and (iii) a tag moiety, and may optionally include one or more linker moieties (e.g., between two other linked moieties).

In some embodiments, a stabilizing moiety and/or a tag moiety may be linked at the N-terminus of the ACE2 moiety, at its C-terminus, or internally (without disrupting relevant activity(ies) of the ACE2 moiety). In some embodiments, stabilizing and tag moieties may be linked at different such positions. As noted herein, in some embodiments, linked moieties may be directly conjugated to one another; in other embodiments moieties may be linked with one another by way of a linker which, for example, may be or comprise a polypeptide of, for example, at least about 5, 10, 15, 20, or 25 amino acids, e.g., selected from glycine, serine, and/or alanine, such as the amino acid sequence of SEQ ID NO:14.

In some embodiments, the ACE2 variants disclosed herein are fusion proteins comprising at least a fragment of ACE2 fused to an exogenous polypeptide as disclosed herein (e.g., ABD, ABDCon, Fc). As such, the molecular weight of the exogenous protein will increase the molecular weight of the fragment of ACE2. In some embodiments, the disclosed fusion proteins, whether glycosylated or non-glycosylated, may have a molecular weight of less than about 250 kD, 225 kD, 200 kD, 175 kD, 150 kD, 125 kD, 100 kD, 95 kd, 90 kD, 85 kD, 80 kD, 75 kD, 70 kD, 65 kD, 60 kD, 55 kD, 50 kD, 45 kD, 40 kD, or 35 kD (or have a molecular weight of a range bounded by any of these values)

ACE2 fusion proteins for use in the disclosed methods and their methods of synthesis are disclosed in the art. (See, e.g., Wysocki et al., “A Novel Soluble ACE2 Variant with Prolonged Duration of Action Neutralizes SARS-CoV-2 Infection in Human Kidney Organoids,” J. Am. Soc. Nephr., 32:795-803, 2021, the content of which is incorporated by reference in its entirety). As disclosed in Wysocki et al., suitable ACE2 fusion proteins for use in the disclosed methods may include ACE2 amino acids 1-618 (SEQ ID NO:10) fused via a linker comprising glycine residues and serine residues to a consensus albumin binding domain (ABDCon) (SEQ ID NO:13). Suitable ACE2 fusion protein may comprise an amino acid sequence as in SEQ ID NO:18.

Modifications

The disclosed ACE2 variants may include an N-terminal methionine residue that does not occur naturally in the native amino acid for ACE2. For example, the amino acid sequence of ACE2 variants contemplated herein may include an N-terminal deletion relative to the amino acid sequence of full-length ACE2, and further, may be modified to include an N-terminal methionine residue that is not present in the amino acid sequence of full-length ACE2.

The disclosed ACE2 variants may be modified so as to comprise an amino acid sequence, or modified amino acids, or non-naturally occurring amino acids, such that the disclosed ACE2 variants cannot be said to be naturally occurring. In some embodiments, the disclosed ACE2 variants are modified and the modification is selected from the group consisting of acylation, acetylation, formylation, lipolylation, myristoylation, palmitoylation, alkylation, isoprenylation, prenylation, and amidation. An amino acid in the disclosed polypeptides may be thusly modified, but in particular, the modifications may be present at the N-terminus and/or C-terminus of the polypeptides (e.g., N-terminal acylation or acetylation, and/or C-terminal amidation). The modifications may enhance the stability of the polypeptides and/or make the polypeptides resistant to proteolysis.

The disclosed ACE2 variants may be modified to replace a natural amino acid residue by an unnatural amino acid. Unnatural amino acids may include, but are not limited to an amino acid having a D-configuration, an N-methyl-α-amino acid, a non-proteogenic constrained amino acid, or a (3-amino acid.

The disclosed ACE2 variants may be modified in order to increase the stability of the ACE2 variants in the target tissue, such as the eye and in particular the cornea. For example, the disclosed peptides may be modified in order to make the peptides resistant to peptidases. The disclosed peptides may be modified to replace an amide bond between two amino acids with a non-amide bond. For example, the carbonyl moiety of the amide bond can be replaced by CH2 (i.e., to provide a reduced amino bond: —CH2-NH—). Other suitable non-amide replacement bonds for the amide bond may include, but are not limited to: an endothiopeptide, —C(S)—NH, a phosphonamide, —P(O)OH—NH—), the NH-amide bond can be exchanged by O (depsipeptide, —CO—O—), S (thioester, —CO—S—) or CH₂ (ketomethylene, —CO—CH₂—). The peptide bond can also be modified as follows: retro-inverso bond (—NH—CO—), methylene-oxy bond (—CH₂—), thiomethylene bond (—CH₂—S—), carbabond (—CH₂—CH₂—), hydroxyethylene bond (—CHOH—CH₂—) and so on, for example, to increase plasma stability of the peptide sequence (notably towards endopeptidases).

The disclosed ACE2 variants may include a non-naturally occurring N-terminal and/or C-terminal modification. For example, the N-terminal of the disclosed peptides may be modified to include an N-acylation or a N-pyroglutamate modification (e.g., as a blocking modification). The C-terminal end of the disclosed peptides may be modified to include a C-amidation. The disclosed peptides may be conjugated to carbohydrate chains (e.g., via glycosylation to glucose, xylose, hexose), for example, to increase plasma stability (notably, resistance towards exopeptidases).

The variants of ACE2 disclosed herein may be further modified. For example, the polypeptide fragment of ACE2 may be further modified to increase half-life in the target tissue and/or to enhance delivery to a target (e.g., cornea of the eye). In some embodiments, the polypeptide fragment is covalently attached to a polyethylene glycol polymer. In other embodiments, the polypeptide fragment may be conjugated to a nanoparticle (e.g., a biogel nanoparticle, a polymer-coated nanobin nanoparticle, and gold nanoparticles). Preferably, the polypeptide fragment of the disclosed methods of treatment and pharmaceutical compositions has a half-live in the target tissue of at least 6 hours, 12 hours, 1 day, 2 days, 3 days, 4 days, 5 days, 6 days, one week, two week, three weeks, four weeks, or longer. Strategies to improve target tissue half-life of peptide and protein drugs are known in the art.

As disclosed in U.S. Pat. No. 10,443,049 (herein incorporated by reference in its entirety), fragments of ACE2 (e.g., SEQ ID NO: 3 and SEQ ID NO: 4) have very high enzymatic activity. Accordingly, such fragments of ACE2 may be utilized in methods of treatment and pharmaceutical compositions as disclosed herein. In some embodiments, the disclosed methods may be practiced in order to reduce AngII (1-8) levels in a subject in need thereof. Moreover, there are substrates other than Angiotensin II that are also cleaved by these ACE2 fragments. Thus, in some embodiments, a pharmaceutical composition is provided, comprising an ACE2 polypeptide, such as SEQ ID NO:1, or a variant or a fragment thereof, such as SEQ ID NO: 3 or SEQ ID NO:4. In some embodiments, methods of treating a subject comprise administering the pharmaceutical composition comprising an ACE2 polypeptide, or a variant thereof.

In some embodiments, the ACE2 polypeptide or variant thereof in the disclosed methods of treatment and pharmaceutical compositions has ACE2 activity for converting AngII (1-8) to Ang (1-7).

In some embodiments, the polypeptide fragment has a molecular weight of less than about 70 kD, 65 kD, 60 kD, 55 kD, 50 kD, 45 kD, 40 kD, 35 kD, 30 kD, 25 kD, 20 kD, 15 kD, 10 kD, 5 kD or less (or have a molecular weight of a range bounded by any of these molecular weight values). The polypeptide fragment may be glycosylated or make lack glycosylation at one or more sites in which full-length ACE2 is glycosylated.

The disclosed polypeptide fragments of ACE2 may include a deletion relative to full-length ACE2 (SEQ ID NO:1). The disclosed polypeptide fragments may include a deletion selected from an N-terminal deletion, a C-terminal deletion, and both, relative to full-length ACE2 (SEQ ID NO:1). Further, in some embodiments the disclosed polypeptide fragments may include an internal deletion. The deletion may remove at least about 1, 2, 3, 4, 5, 10, 15, 20, 25, 50, 100, 150, 200 amino acids or more of full-length ACE. In some embodiments, the deletion removes one or more glycosylation sites, and as such, the polypeptide fragments of ACE2 may be less glycosylated than full-length ACE2, further reducing the molecular weight of the polypeptide fragments of ACE2 relative to full-length ACE2.

Characterization

In some embodiments, a provided soluble ACE2 variant polypeptide agent may be characterized by catalytic efficiency (e.g., cleavage of a substrate such as Ang II-(1-8) and/or des-9Arg-Bradykinin (bradykinin-(1-8), which may, in some embodiments, be determined, for example via a phenylalanine assay as described herein) that is comparable to that observed with an appropriate reference ACE2 polypeptide (e.g., a parent soluble ACE2(1-740)).

In some embodiments, a provided soluble ACE2 variant polypeptide agent may be characterized by blood pressure-lowering effect during acute Ang II infusion (e.g., as may be assessed in a rodent such as a mouse) that is comparable to that observed with an appropriate reference ACE2 polypeptide (e.g., a parent soluble ACE2(1-740)).

In some embodiments, a provided soluble ACE2 variant polypeptide agent may be characterized by increased serum Cmax when compared with an appropriate reference ACE2 polypeptide (e.g., a parent soluble ACE2(1-740)). In some embodiments, a provided soluble ACE2 variant polypeptide agent may be characterized by a serum Cmax that is approximately twice that of such reference, or even more.

In some embodiments, a provided soluble ACE2 variant polypeptide agent may be characterized by prolonged in vivo activity when compared with an appropriate reference ACE2 polypeptide (e.g., a parent soluble ACE2(1-740)). In some embodiments, an assessed in vivo activity may be or comprise a binding activity (e.g., a spike protein binding activity), a neutralization activity (e.g., inhibition of infection), an enzymatic activity (e.g., ACE2 enzymatic activity, such as may be assessed, for example, by direct cleave assay and/or by ability to reduce blood pressure during acute Ang II infusion).

In some embodiments, a provided soluble ACE2 variant polypeptide agent may be characterized by an observed in vivo activity persistence (i.e., detectable activity above an established threshold) in a relevant assay that is at least approximately 5, 10, 15, 20, 25, 30, 35, 40, 45, 50, 55, 60, 65, 70, 75, 80, 85, 90, 95, 100 or more times that of an appropriate reference ACE2 polypeptide (e.g., a parent soluble ACE2(1-740)); alternatively or additionally at least 10, 20, 30, 40, 50, 60, 70, 80, 90, 100, 110, 120, 130, 140 days longer. In some embodiments, a provided soluble ACE2 variant polypeptide agent is characterized by persistence of one or more activities in vivo above a relevant threshold for at least about 1, 2, 3, 4, 5, 6, 7, 8, 9, 10 days or more after administration of the soluble ACE2 variant polypeptide agent, or, in some embodiments at least about 1, 2, 3, 4, 5, 6, 7, 8, 9, 10, 11, 12, 13, 14, 15, 16, 17, 18, 19, 20 weeks or more; in some embodiments, 1, 2, 3, 4, 5, 6 months or more, etc.

In some embodiments, a provided soluble ACE2 variant polypeptide that demonstrates persistence of in vivo activity as described herein may be amenable to dosing according to a regimen that includes only a single dose or a small number of doses and/or in which doses may be separated from one another by a relatively extended period of time (e.g., reflective of time of activity persistence in vivo).

In some embodiments, a soluble ACE2 variant polypeptide is characterized relative to a particular reference. To give but a few examples, in some such embodiments, a reference may be or comprise ACE2(1-740) (e.g., hACE2(1-740)). Alternatively or additionally, in some embodiments, a reference may be or comprise hACE2(1-618) and/or mACE2(1-619)(which may, in some embodiments, be particularly useful as a negative control). Further alternatively or additionally, in some embodiments, an agent that is specifically exemplified herein (e.g., hACE2(1-618)-ABD and/or hACE2(1-618)-Fc, etc) may be utilized as a reference. That is, the present disclosure having identified and/or documented particular attributes of such exemplified agents, and thus having demonstrated feasibility of their performance, the present disclosure further teaches their use as comparators for development or confirmation of additional useful agents, including alternative soluble ACE2 variant polypeptide agents within the scope of the present disclosure.

Pharmaceutical Compositions

Subjects suitable for the disclosed methods of treatment may include, but are not limited to, subjects having or at risk for developing disease or conditions that negatively affect the cornea. In some embodiments, the corneal disease or conditions result in one or more of corneal deformation, corneal clouding, corneal inflammation, corneal neovascularization, corneal edema, and/or the increased expression of one or more of markers of inflammation, AngII, and ROS.

The compositions disclosed herein may include pharmaceutical compositions comprising an ACE2 polypeptide, variants and/or fragments thereof, and may be formulated for administration to a subject in need thereof. Compositions may include one, or more than one, different ACE2 polypeptide and/or variant(s) (e.g., a composition may include one or more of SEQ ID NO:1, SEQ ID NO; 3, SEQ ID NO:4, SEQ ID NO:10, and SEQ ID NO:18). Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”)).

In some embodiments, the pharmaceutical compositions are formulated for extraocular delivery, e.g., topical delivery or periocular delivery including subconjunctival delivery and sub-tenon delivery. In some embodiment, the compositions are formulated for intraocular delivery, including intrastromal delivery, intraviteral delivery, and suprachordial delivery. In some embodiments, compositions are formulated for systemic delivery, such as oral or parenteral delivery.

In some embodiments, compositions comprising an ACE2 polypeptide or variants thereof, are formulated as ophthalmic compositions for extraocular (e.g., topical) delivery. Ophthalmic compositions are used to provide relief of a variety of ocular conditions and ocular disease states (e.g., diseases and conditions affecting the cornea). Often, ophthalmic compositions are administered or instilled to the eye via eye drops from a multi-dose container in the form of solutions, suspensions, ointments or gels. If the ophthalmic active component (e.g., an ACE2 polypeptide, variants and/or fragments thereof) is sufficiently soluble in water, the formulation may have the form of a solution eye drop product. However, if the solution product has too low of a viscosity, e.g., less than about 30 cp (or mPa s), upon instillation the ophthalmic active can be rapidly discharged from the pre-corneal area of the eye because of lacrimal secretion and nasolacrimal drainage. As a result, it has been estimated that approximately 80-99% of the ophthalmic active component is simply washed or flushed from the eye before the active actually contacts the desired ocular tissue to achieve its desired clinical effect. The poor residence time of the active in the eye thus requires frequent instillation or use of a more concentrated active product to achieve the desired clinical effect. To lengthen the residence time of ophthalmic active (e.g., an ACE2 polypeptide, variants and/or fragments thereof), and thus, to enhance the bioavailability of the ophthalmic active per instillation, non-solution based ophthalmic vehicles may be employed. Examples of such ophthalmic vehicles include ointments, suspensions, and aqueous gels.

In some embodiments, ophthalmic vehicles have the form of the so-called in situ gel-forming systems. These ophthalmic vehicles can extend pre-corneal residence time and improve ocular bioavailability of the ophthalmic active. Typically, in situ gel-forming systems are aqueous solutions containing a polymer system. The ophthalmic products tend to exist as a low-viscosity liquid during storage in the dispenser container and form a gel upon contact with tear fluid. The liquid-to-gel transition can be triggered by a change in temperature, pH, ionic strength, or the presence of tear proteins, depending on the particular polymer system employed. Such a gel can have an extended residence in the eye and assist in promoting a higher drug bioavailability, and perhaps enhance clinical outcome per instillation.

In some formulations, the ophthalmic active (e.g., an ACE2 polypeptide, or variant thereof) is in a form that is virtually, or completely, insoluble in an aqueous solution-based formulation. For example, U.S. Pat. Nos. 5,538,721 and 4,540,930 describe a pharmaceutical composition comprising an amino-substituted steroid therapeutic agent, and an effective stabilizing amount of lightly cross-linked carboxy-containing polymer. Cyclodextrin has also been used to at least partially solubilize the therapeutic agent in an aqueous medium.

Lotemax™ (loteprednol etabonate (LE) ophthalmic gel, 0.5% LE) (Bausch & Lomb Incorporated) contains 5 mg/g of loteprednol etabonate, as a sterile preserved ophthalmic gel suspension, and has proven effective for the treatment of post-operative inflammation and pain following ocular surgery. Lotemax™ ophthalmic gel, 0.5% LE, contains boric acid, edetate disodium dihydrate, glycerin, polycarbophil, propylene glycol, sodium chloride, tyloxapol, water, and sodium hydroxide to adjust pH between 6 and 7, and is preserved with benzalkonium chloride (BAK) 0.003%. In some embodiments, a composition comprising Lotemax and an ACE2 polypeptide, or variants thereof, is provided.

DUREZOL™ (difluprednate ophthalmic emulsion 0.05%) (Alcon Laboratories, Inc.), a sterile preserved ophthalmic emulsion for topical ophthalmic administration, has proven effective for the treatment of inflammation and pain associated with ocular surgery, and is also indicated for the treatment of endogenous anterior uveitis. DUREZOL™ ophthalmic emulsion contains difluprednate (0.05%), boric acid, castor oil, glycerin, sodium acetate, sodium EDTA, sodium hydroxide to adjust pH, polysorbate 80 and water, and is preserved with sorbic acid 0.1%. In some embodiments, a composition comprising DUREZOL and an ACE2 polypeptide, or variants thereof, is provided.

In some embodiments, the ophthalmic formulation comprises a suspending agent and a non-ionic cellulose derivative. The suspending agent may comprise a carboxyvinyl polymer, such as polycarbophil or carbomer. The non-ionic cellulose derivative may be hydroxypropylmethyl cellulose.

The ophthalmic formulation may include, in addition to an ACE2 polypeptide, or variants thereof, one or more additional active agents. By way of example, the one or more active agents may include an antibiotic, anti-inflammatory agent, a steroid, or a non-steroidal anti-inflammatory drug. For example, an additional active agent may include a corticosteroid, such as loteprednol etabonate or difluprednate, or a non-steroid, such as nepafenac. In some embodiments, additional active agents may include one or more of anti-VEGF agents, such as, but not limited to pegatanib (Macugen®), ranibizumab (Lucentis®), aflibercept (Eylea®) that serves as “VEGF trap”, and bevacizumab (Avastin®); anti-TNFα agents such as, but not limited to Adalimumab, infliximab; GLP-1 agonists such as but not limited to Exenatide (Byetta®/Bydureon®), Liraglutide (Victoza®/Saxenda®), albiglutide (Tanzeum®) and Dulaglutide (Trulicity®); other protein or peptide based therapies such as but not limited to Abicipar pegol, Pegpleranib (Fovista®, Ophthotec), Nesvacumab (Regeneron), and Zimura® (Ophthotec).

The formulation may further comprise a preservative and/or a surfactant.

According to various aspects, the formulation may comprise one or more of polycarbophil, hydroxypropylmethyl cellulose, benzalkonium chloride, a poloxamer surfactant, glycerin, propylene glycol, a borate buffer agent; small sugars (e.g. trehalose) and polysaccharides (e.g. dextrans) may be included to enhance the stability of the peptides. Pluronics and non-ionic surfactants such as polysorbates at low concentrations may be included to decrease peptide aggregation.

In some embodiments, the ophthalmic composition comprises a suspension having the form of a gel at room temperature and that forms a liquid upon instillation in an eye, in particular, the cornea of the eye.

According to various aspects, an ACE2 polypeptide, or variant thereof, and the one or more ophthalmic active agents may be present in the composition as particles or may be soluble. By way of example, in some embodiments, micro particles or microspheres may be employed, e.g., by utilizing one or more of poly(lactic-co-glycolic acid) (PLGA), polyanhydrides and cyclodextrins. Nanoparticles may also be employed, e.g., by utilizing biodegradable polymers and lipids to form liposomes, dendrimers, micelles, or nanowafers as carriers for targeted delivery of the ACE2 polypeptide or variant thereof. In some embodiments, polymeric implants may be used.

In some embodiments, the composition if formulated for ocular administration, and comprises between 0.1 ng and 500 mg/ml of the ACE2 peptide, or variant thereof. In some embodiments, the compositions if formulated such that between 0.1 ng and 500 μg of the ACE2 peptide, or variant thereof is administered to an eye of a subject (e.g., to the cornea). In some embodiments, the composition is formulated such that between about 10 fmol and 500 pmol is administered to an eye of the subject (e.g., to the cornea). In some embodiments, the composition is formulated such that between about 0.1 and 5 mg/kg body weight is administered to an eye of the subject (e.g., to the cornea).

Corneal Eye Diseases and Conditions

Provided herein are compositions and methods useful to treat disease and conditions of the cornea. In some embodiments, the corneal disease or conditions result in one or more of clouding of the cornea, inflammation of the cornea, deformation of the cornea, neovascularization of the cornea, edema in the cornea, increased expression of inflammatory markers in the cornea, increased expression of Ang II in the cornea, increased expression of ROS in the cornea. In some embodiments, the corneal condition includes trauma or injury.

Inflammatory markers include, but are not limited to interleukins (IL-1a, IL-1b), chemokines (CCL2, CLCX8), TNF-α, and CD11c, CD68, and CD3 positive cells.

The cornea, as the most anterior structure of the eye, is exposed to various hazards ranging from airborne debris to blunt trauma that can result in mechanical trauma. The cornea and anterior surface of the eye can also be exposed to other forms of trauma from surgery, and chemical, such as acid and alkali, injuries. Additional types of injury or trauma include, e.g., laceration, chemical burns, welding injuries, or foreign body, laceration. The results of these types of injuries can be devastating often leading to corneal and conjunctival scarring symblephera formation. In addition, corneal neovascularization may ensue. Neutrophils accumulate, their release of leukotrienes, and the presence of interleukin-1 and interleukin-6, serves to recruit successive waves of inflammatory cells (Sotozono, et al. (1997), Curr Eye Res, vol 19: 670-676) that infiltrate the cornea and release proteolytic enzymes, which leads to further damage and break down of corneal tissue and a corneal melt. In addition, corneal and conjunctival fibroblasts become activated and invade and leading to collagen deposition and fibrosis. The undesirable effects of excessive inflammation and scarring are promoted by TGFβ Saika, et al. (2006), Am J Pathol vol 168, 1848-60. This process leads to loss of corneal transparency and impaired vision. Reduced inflammation, including decreased neutrophil infiltrates and reduced fibrosis resulted in faster and more complete healing in a murine model of alkali burned corneas (Ueno, et al. (2005), Opthalmol Vis Sci, vol 46: 4097-106).

In addition to trauma, diseases and conditions exist that negatively impact the cornea and result in one or more of corneal deformation, corneal clouding, corneal inflammation, corneal neovascularization, and/or the increased expression of markers of inflammation, and/or the increased expression of ROS, and/or the increased expression of AngII. Such diseases and conditions include, but are not limited to bullous keratopathy, pterygium, corneal ulcer, herpes simplex keratitis, herpes zoster ophthalmicus, herpes zoster keratitis, fungal keratitis, interstitial keratitis, keratoconjunctivitis sicca, keratomalacia, peripheral ulcerative keratitis, phlyctenular keratoconjunctivitis, superficial punctate keratitis, keratoconus, corneal dystrophies such as, but not limited to Fuch's Dystrophy, Lattice Dystrophy Type I, Lattice Dystrophy Type II, congenital stromal corneal dystrophy, Meesmann corneal dystrophy, and map dot fingerprint dystrophy, congenital corneal diseases such as, but not limited to complete LCAT deficiency, fish-eye disease, keratitis-ichthyosis-deafness syndrome, Peters anomaly, Peters plus syndrome; Bowen disease, Cogan syndrome, dry eye, dryness in the eye due to Sjogren syndrome, vitamin A deficiency, or LASIK eye surgery, infections, sensitivity to non-infectious bacteria or toxins, allergies, trachoma, river blindness, corneal transplant, recurrent corneal erosion, and tumors.

Methods

Disclosed herein are methods of treating an ophthalmic condition that comprises administering to a patient in need thereof, a pharmaceutical composition comprising an ACE2 polypeptide, or a fragment or variant thereof. In some embodiments, the subject is diagnosed or is at risk of developing a disease or condition that negatively impacts the cornea. In some embodiments, the subject's cornea exhibits one or more of an injury, deformation, inflammation, clouding, neovascularization, edema, increased expression of a marker of inflammation, ROS, or AngII. In some embodiments, the subject is diagnosed with or is at risk of developing a disease or condition, such as, but not limited to injuries such as, but not limited to corneal laceration, chemical burns, welding injuries, or foreign body, or disease such as but not limited to bullous keratopathy, pterygium, corneal ulcer, herpes simplex keratitis, herpes zoster ophthalmicus, herpes zoster keratitis, fungal keratitis, interstitial keratitis, keratoconjunctivitis sicca, keratomalacia, peripheral ulcerative keratitis, phlyctenular keratoconjunctivitis, superficial punctate keratitis, keratoconus, corneal dystrophies such as, but not limited to Fuch's Dystrophy, Lattice Dystrophy Type I, Lattice Dystrophy Type II, congenital stromal corneal dystrophy, Meesmann corneal dystrophy, and map dot fingerprint dystrophy, congenital corneal diseases such as, but not limited to complete LCAT deficiency, fish-eye disease, keratitis-ichthyosis-deafness syndrome, Peters anomaly, Peters plus syndrome; Bowen disease, Cogan syndrome, dry eye, dryness in the eye due to Sjogren syndrome, vitamin A deficiency, or LASIK eye surgery, infections, sensitivity to non-infectious bacteria or toxins, allergies, trachoma, river blindness, corneal transplant, recurrent corneal erosion, and tumors. In some embodiments, the methods are useful to treat inflammation in the cornea of a subject in need thereof.

In some embodiments, the composition is formulated as an ocular composition and is administered directly to the eye of the subject, e.g., as eye drops. In some embodiments, the composition is self-administered.

In some embodiments, the composition is formulated for extraocular delivery, e.g., topical delivery, periocular delivery including subconjunctival delivery or sub-tenon delivery. In some embodiment, the composition is formulated for intraocular delivery, including intrastromal delivery, intraviteral delivery, and suprachordial delivery. In some embodiments, the composition is formulated for systemic delivery, such as oral or parenteral delivery. In some embodiments, minimally invasive microneedles and/or iontophoresis may be used to administer the composition.

In some embodiments, the composition is administered once per day; in some embodiments, the composition may be administered multiple times per day, e.g., at a frequency of one or two times per day, or at a frequency of three or four times per day or more.

In some embodiments, the composition is administered such between about 0.1 ng and 500 mg/ml of the ACE2 peptide, or variant thereof reaches an eye of the subject (e.g., at the cornea). In some embodiments, the composition is administered such between about 0.1 ng and 500 μg of the ACE2 peptide, or variant thereof reaches an eye of a subject (e.g., at the cornea). In some embodiments, the composition is administered such between about 10 fmol and 500 pmol reaches an eye of the subject (e.g., at the cornea).

In some embodiments, the treatment reduces the symptoms of the disease or condition more quickly than if no treatment is provided for the same or similar disease or condition. By way of example, in some embodiments, a decrease in one or more of corneal cloudiness, inflammation, deformation, neovascularization, and edema is observed more quickly than if no treatment is provided for the same or similar condition or disease. In some embodiments, inflammation, and/or a decrease in one or more markers of inflammation, such as interleukins (IL-1a, IL-1b), chemokines (CCL2, CLCX8), TNF-α, and CD11c, CD68, and CD3 positive cells is observed more quickly than if no treatment is provided for the same or similar condition or disease. In some embodiments in which corneal injury/trauma is involved, wound healing is observed more quickly than if no treatment is provided for the same or similar injury. In some embodiments, a decrease in AngII and/or ROS levels is observed more quickly than if no treatment is provided for the same or similar condition or disease.

In some embodiments, improvements in the condition of the subject's cornea is observed more quickly than if no treatment is provided for the same or similar condition or disease. By way of example, in some embodiments, improvements in the condition of the subject's cornea is observed within 24 hours of administration of the composition. In some embodiments, improvements in the condition of the subject's cornea is observed within about 1 to about 3 days; within about 3 to about 5 days, or within about a week of the first administration. In some embodiments, improvements in the condition of the subject's cornea is observed within about 10 days, about 14 days or within about 1 month of the first administration.

### EXAMPLES

The following examples are illustrative and should not be interpreted to limit the scope of the claimed subject matter.

### Example 1: ACE2 as a Therapeutic in Diseases and Conditions of the Cornea

Abstract

Angiotensin converting enzyme 2 (ACE2) plays an important role in inflammation, which is attributable at least, in part, to the conversion of the proinflammatory angiotensin (Ang) II peptide into angiotensin 1-7 (Ang 1-7), a peptide which opposes the actions of AngII. ACE2 and AngII are present in many tissues but information on the cornea is lacking. We observed that mice deficient in the Ace2 gene (Ace2^{-/-}), developed a cloudy cornea phenotype as they aged. Haze occupied the central cornea, accompanied by corneal edema and neovascularization. In severe cases with marked chronic inflammation, a cell-fate switch from a transparent corneal epithelium to a keratinized, stratified squamous, psoriasiform-like epidermis was observed. The stroma contained a large number of CD11c, CD68, and CD3 positive cells. Corneal epithelial debridement experiments in young ACE2-deficient mice showed normal appearing corneas, devoid of haze. We hypothesized, however, that these mice are “primed” for a corneal inflammatory response, which once initiated, would persist. In vitro studies reveal that interleukins (IL-1a, IL-1b), chemokines (CCL2, CLCX8), and TNF-α, are all significantly elevated, resulting in a cytokine storm-like phenotype. This phenotype could be partially rescued by treatment with the AngII type 1 receptor (AT1R) antagonist, losartan, suggesting that the observed effect was mediated by AngII acting on its main receptor.

Results

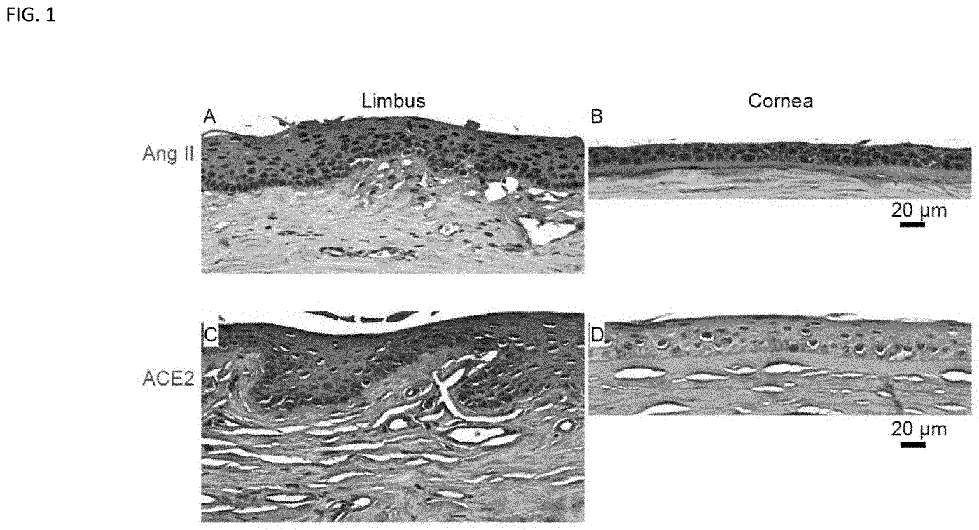

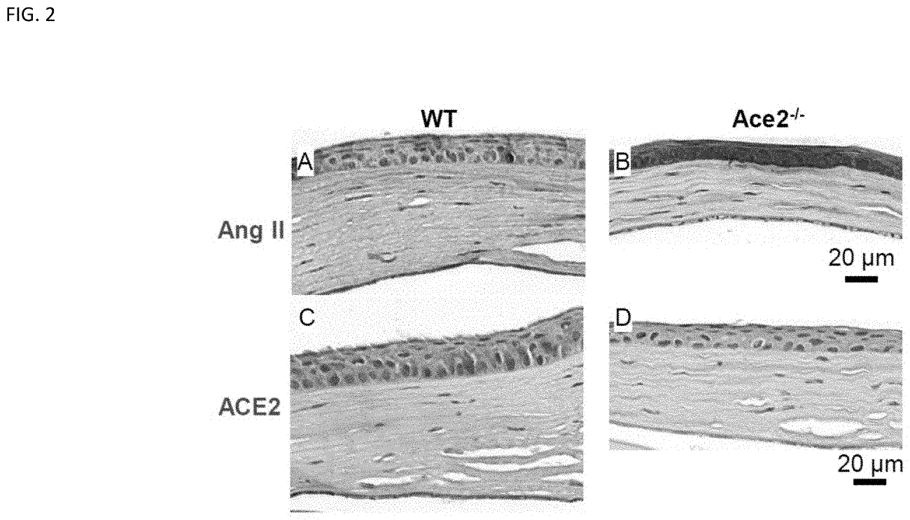

AngII and Ace2 are expressed in limbus and cornea. Immunohistochemical analysis revealed that the expression of AngII was strong throughout the human limbal and corneal epithelia as well as the stromal vessels (FIG. 1a, b). In contrast, ACE2 expression was prominent in the limbal basal cells and vasculature (FIG. 1c). AngII and ACE2 were detected throughout the wild-type (WT) mouse corneal epithelium (FIG. 2a, c) but AngII appeared to be more patchy in its distribution (FIG. 2a). In the ACE2-deficient mice (Ace2^{-/-}), no ACE2 was detected in the corneal epithelium (FIG. 2d) whereas AngII expression was markedly increased (FIG. 2b) compared with the WT (FIG. 2a).

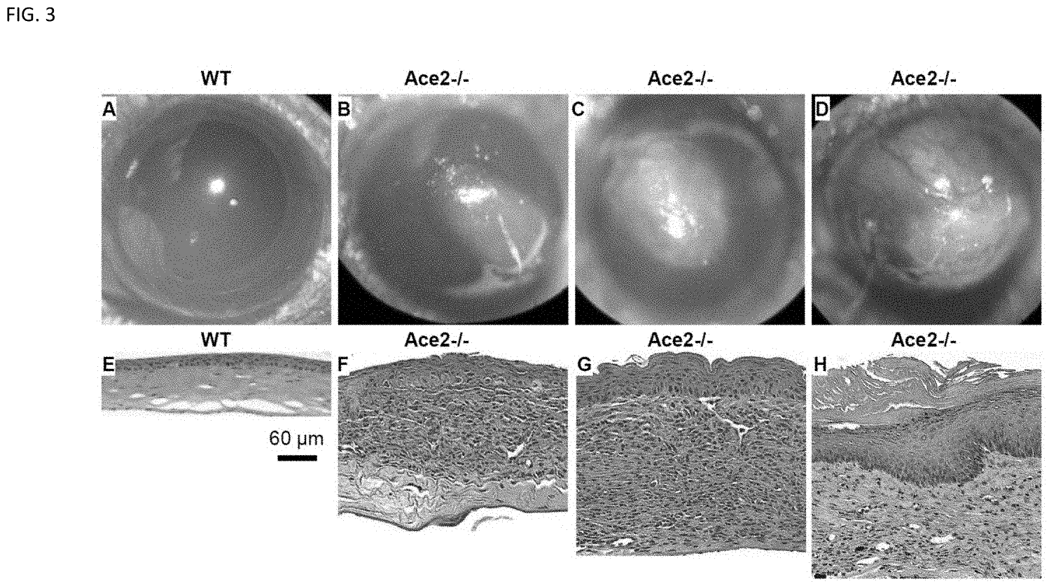

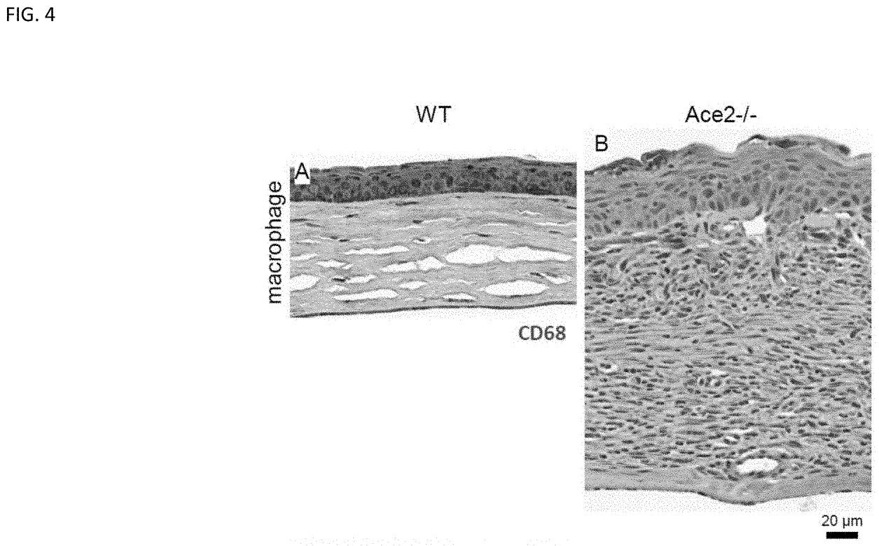

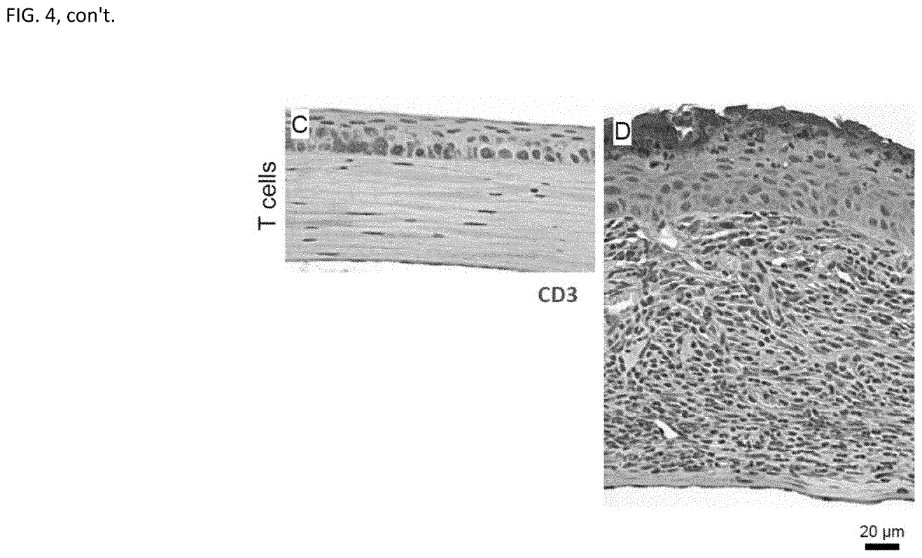

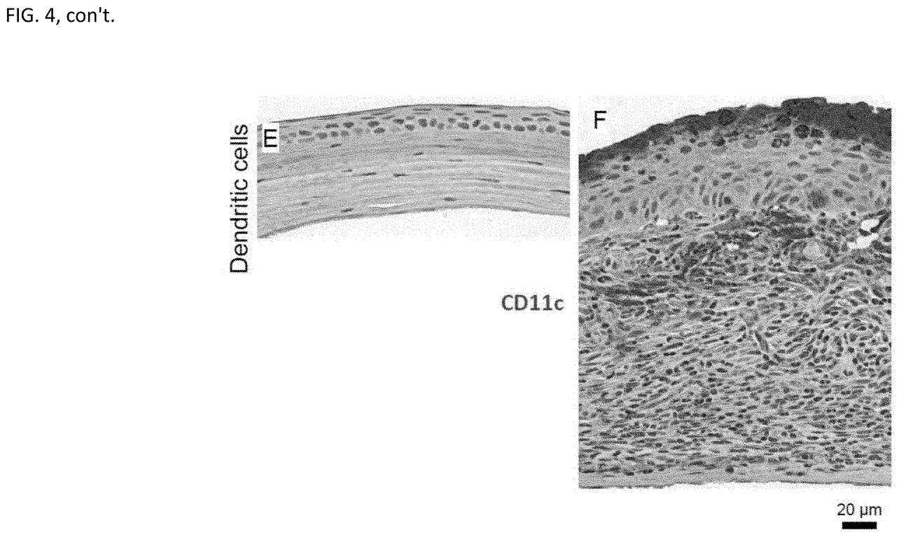

Loss of ACE2 results in marked corneal changes. Aged Ace2^{-/-} mice developed striking changes in corneal epithelial morphology as well as significant stromal angiogenesis and an inflammatory infiltrate (FIG. 3). Interestingly these changes appeared in approximately 70% of adult (>1 year old) mice. In many instances, these changes only occurred in one eye. Those Ace2^{-/-} mice that developed inflammation were readily recognized clinically by a cloudy, hazy cornea (FIGS. 3b-d). A wide spectrum of corneal epithelial alterations were detected, ranging from a thin, disorganized epithelium (FIG. 3f) to a more stratified, thickened epidermoid tissue (FIG. 3h). In all eyes examined, the stroma consisted of a densely packed admixture of polymorphonuclear, mononuclear, and lymphocytic infiltrates along with numerous vascular profiles (FIGS. 3f-h). On higher magnification, the infiltrates were identified as neutrophils by H&E, and as macrophages, T cells and dendritic cells identified by immunohistochemistry staining for CD68, CD3 and CD11c cells, respectively (FIG. 4).

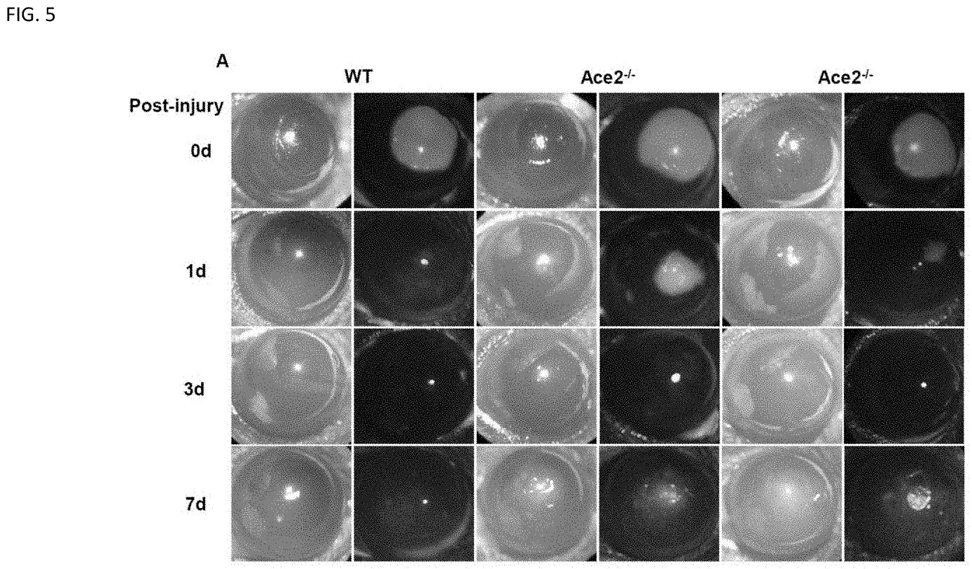

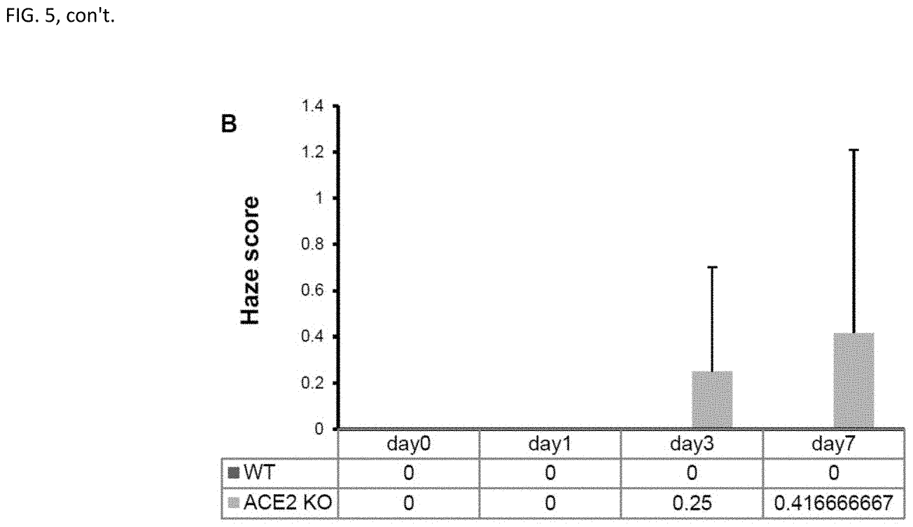

Ace2^{-/-} mice are “primed” to develop marked inflammation in response to a minor stress. Since in 30% of Ace2^{-/-} mice, the cloudy corneas were not bilateral, this would suggest that the lesions are a direct sequelae of external perturbation rather than arising from a systemic deficiency. To test this idea, we made small 1 mm circular debridement wounds in young Ace2^{-/-} mice with clear corneas and compared the response to aged-matched wild-type littermates. In this well adopted procedure, the type of wound removes the corneal epithelium but does not result in a significant inflammatory infiltrate¹⁰. Immediately following wounding, all mice received topical application of a 0.5% fluorescein stain, and the rate of epithelial healing was visualized. As expected, WT mice sealed wounds within 24 hours (FIG. 5a) whereas the Ace2^{-/-} mice still had detectable fluorescein staining at this time (FIG. 5a). Three days post-wounding, WT mice had clear corneas devoid of fluorescein staining. Similarly, the Ace2^{-/-} mice had minimal fluorescein staining three days post wound; however, there was development of haze prompting further clinical evaluation of corneal clarity¹¹ (FIG. 5b). Day 7 post-wounding, WT mice appeared normal, whereas the Ace2^{-/-} mice still had cloudy corneas and patches of fluorescein staining detected on the surface, suggestive of a defective barrier.

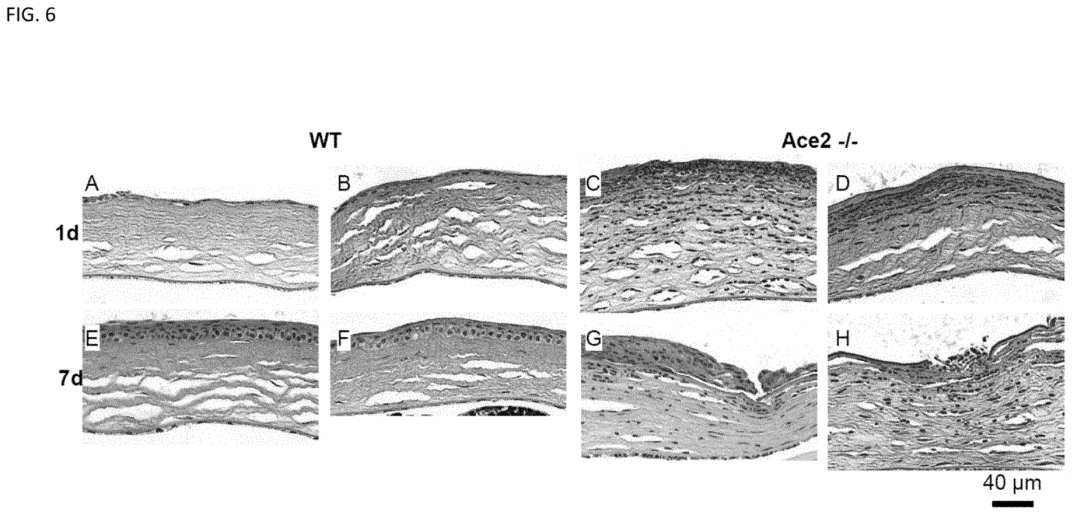

One day post-wounding, the WT mice had completely re-epithelialized the surface as evidenced by a thin, 1-2 cell layered, well-organized epithelium (FIG. 6a, b). This was contrasted by the Ace2^{-/-} mice, which were characterized by a single, thin layer of disorganized epithelial cells, lacking evidence of stratification (FIG. 6c d). At day one post-wounding, the stroma of the WT mice appeared normal consisting of a plywood-like organization of collagen bundles, interspersed with numerous keratocytes. Few if any inflammatory cells were noted (FIG. 6a, b). The stroma of the Ace2^{-/-} mice was filled with inflammatory cells (primarily polymorphonuclear cells) and numerous vascular profiles, characteristic of a brisk inflammation (FIG. 6c, d). Seven days post-wounding, WT mice had a multi-layered, stratified epithelium characteristic of an unwounded mouse (FIG. 6e, f) overlying a normal appearing stroma devoid of inflammatory cells. The corneal epithelium from Ace2^{-/-} mice, seven days post wounding was variable in appearance. For example, some mice had a markedly thickened multi-layered epithelium (FIG. 6g), whereas some mice had a thin, single-layered epithelium (FIG. 6h). In all cases the Ace2^{-/-} mice, seven days post wounding, had a stroma with variable amounts of inflammatory cells and vascular remnants (FIG. 6g, h).

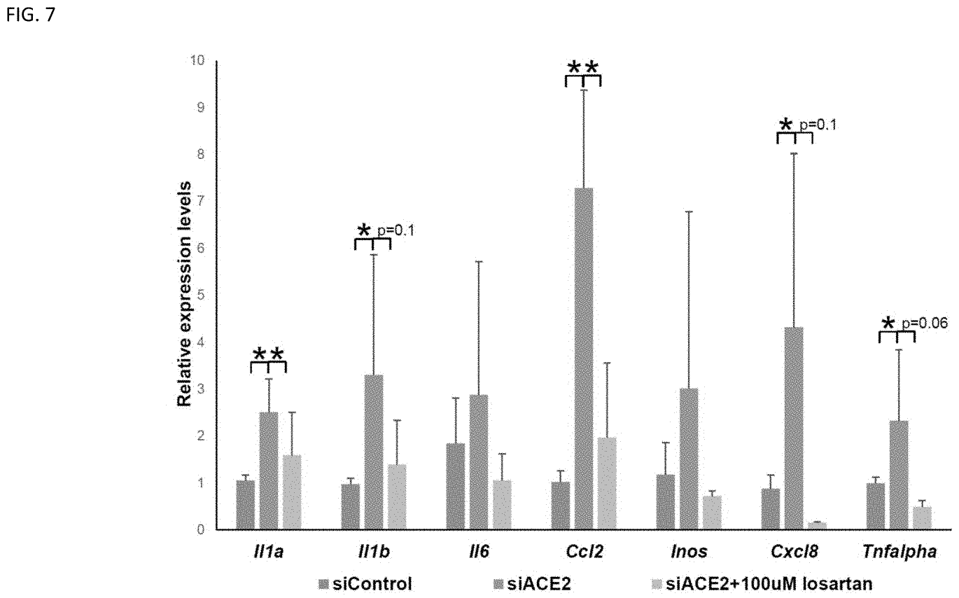

Induction of cytokine expression in epithelial cells lacking Ace2 contribute to the primed inflammatory response in the Ace2^{-/-} mice. Following a perturbation such as wounding, epithelial cells produce pro-inflammatory cytokines, which are known to initiate the inflammatory response¹². To investigate the mechanism of this initial phase of inflammation in the context of ACE2, we knocked down ACE2 in a corneal epithelial cell line (hTCEpi) and evaluated the expression of several cytokines. Knockdown of ACE2 in hTCEpi cells with an siRNA smartpool increased the expression of ILIA, IL1B, IL6, TNFA, CCL2, CXCL8, and INOS compared to the siRNA control (FIG. 7).

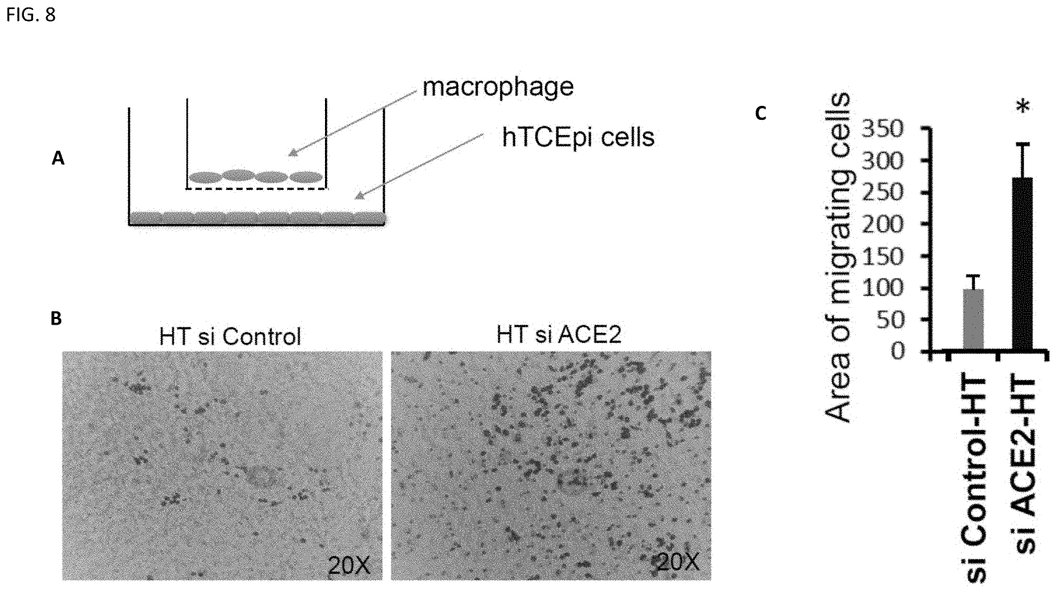

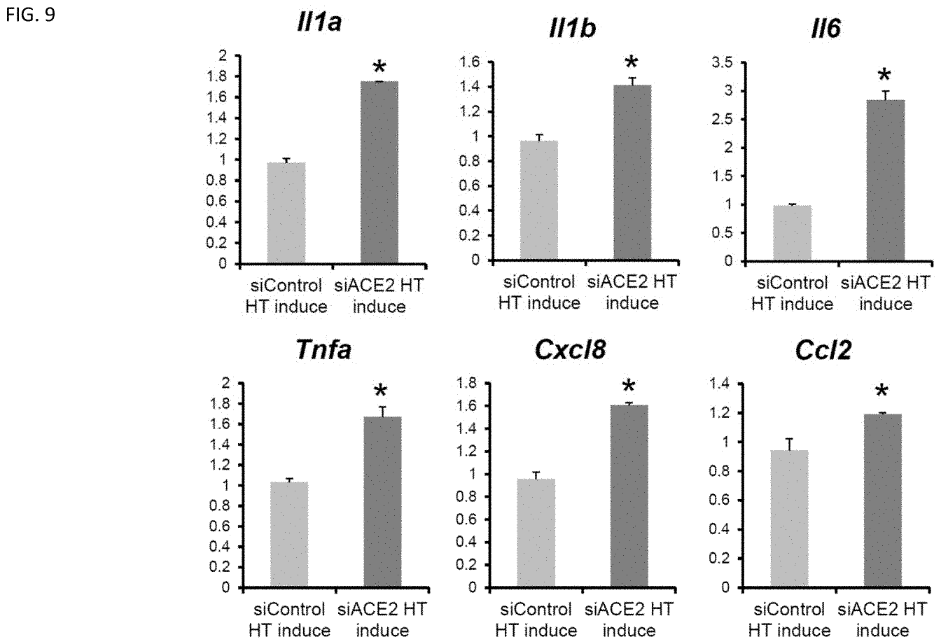

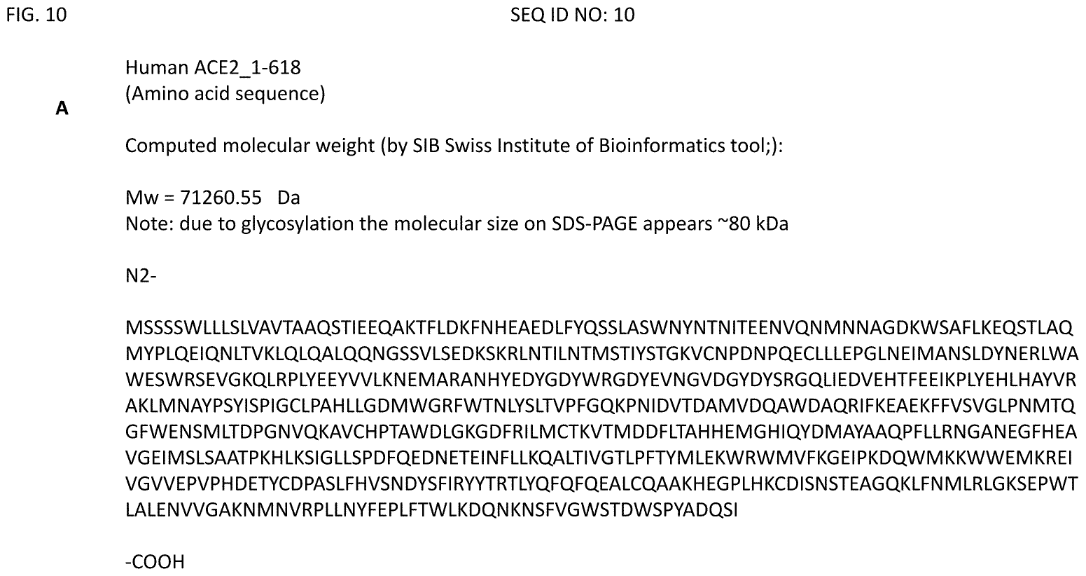

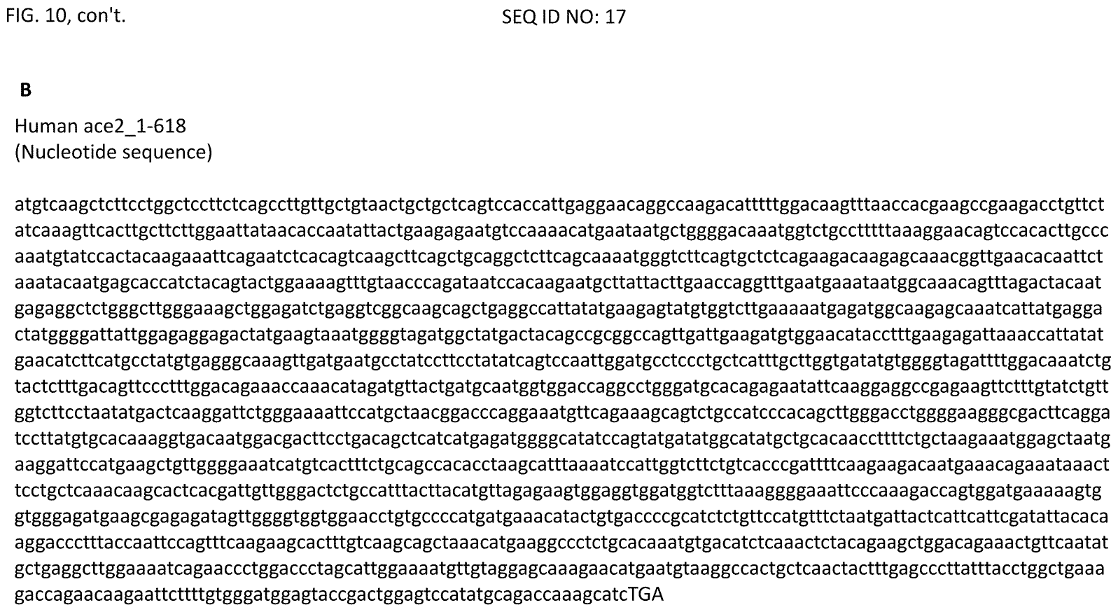

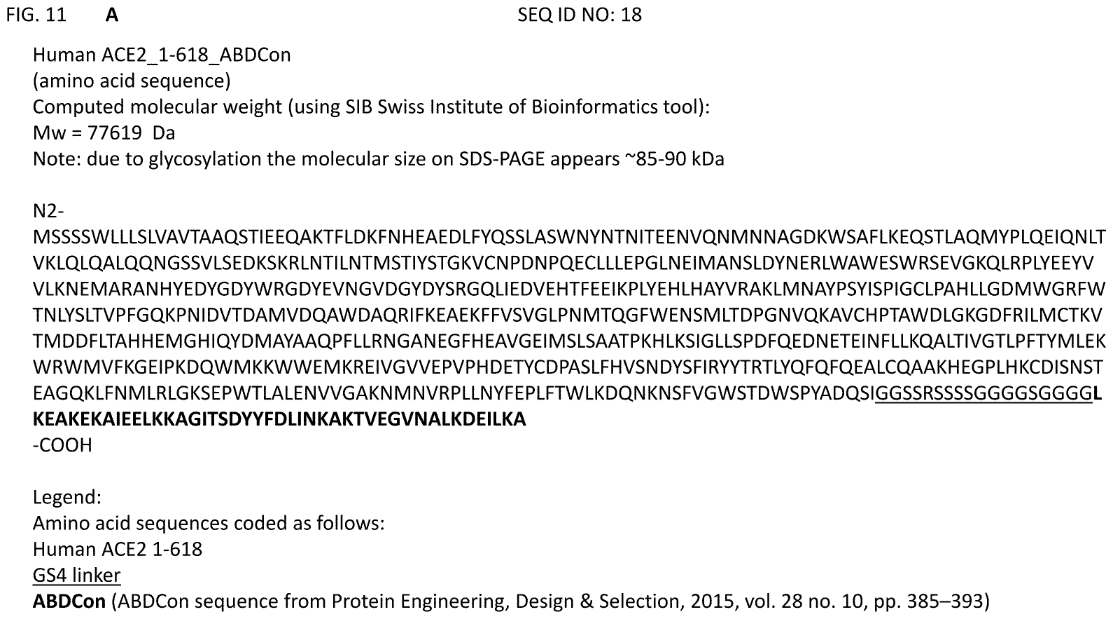

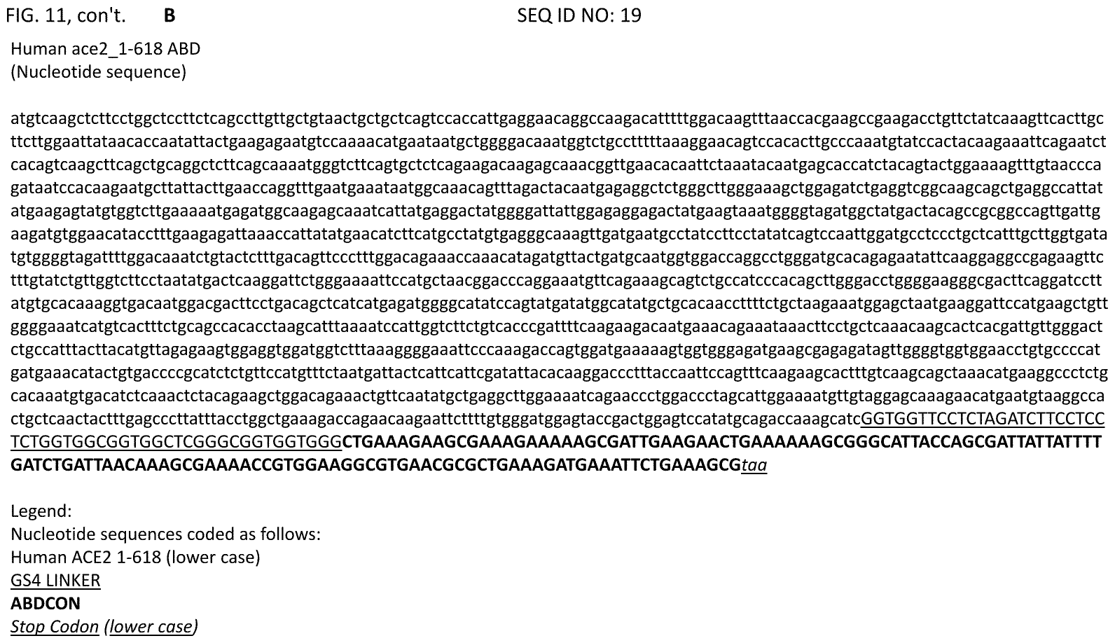

To explore whether such an increase in cytokine expression in corneal epithelial cells enhanced recruitment of immune cells, we conducted trans-well chemotaxis assays using a modified Boyden chamber co-cultured with the RAW-Dual™ macrophage cell line against hTCEpi cells on the bottom chamber. Increased migration of macrophages was observed when ACE2 was knocked-down in hTCEpi cells (FIG. 8). This suggests that ACE2 deficiency was at least in part responsible for the recruitment of macrophages into the inflammatory milieu. The increases observed in CCL2 and TNFA are associated with the recruitment and activation of macrophages^{13, 14, 15, 16}. Therefore, we explored the possibility that ACE2-deficient epithelial cells could activate macrophages. We exposed a PMA-stimulated THP-1-Dual™ human monocytic cell line to conditioned media from hTCEpi cells lacking ACE2. Such treatment significantly increased the expression of ILIA, IL1B, IL6, TNFA, and CXCL8 compared with control hTCEpi conditioned media (FIG. 9). These findings indicate that loss of ACE2 in hTCEpi can activate macrophages.

To examine if ACE2 deficiency-induced cytokine production is due to an increase in AngII activity, we investigated whether treatment of corneal epithelial cells with the AngII type 1 receptor (AT1R) antagonist, losartan could rescue the phenotype. hTCEpi deficient in ACE2 were treated with 100 μm losartan for 24 hrs and the cytokine response was evaluated. Losartan treatment decreased the expression of ILIA, TNFA, and CCL2 (FIG. 7). This suggests that ACE2 deficiency is responsible for the excessive expression of cytokines in the inflammatory milieu via the activation of AngII.

Discussion

RAS components are found in ocular tissues of various species, as well as humans. The major components including renin, angiotensinogen, and ACE2 have been identified in the retina, ciliary body, vitreous fluid, iris, choroid, aqueous fluid, sclera, and conjunctiva (¹⁷ and references therein). Interestingly, little if any attention has been focused on defining RAS in the limbus/cornea. We now demonstrate that ACE2 is present in both human and mouse limbal and corneal tissues and that the genetic deficiency of ACE2 results in a marked inflammatory response in corneal epithelial and stromal tissues. Moreover, the deficiency of ACE2 resulted in marked upregulation of AngII, the main peptide that is degraded normally by ACE2.

As ACE2-deficient mice aged, more than 70% developed cloudy corneas in either one or both eyes. This led us to postulate that external stress (e.g. scratching or wounding) to the corneal surface resulted in an inflammatory event that could not be resolved due to the inability to regulate AngII. To test this concept, we induced gentle debridement of the corneal epithelium, which in WT mice does not result in a significant inflammatory response¹⁰. In the Ace2^{-/-}, this procedure caused a significant inflammatory response in young mice with otherwise normal appearing corneas (FIGS. 5, 6). This strongly supports the idea that Ace2^{-/-} mice are “primed” for an inflammatory response and results in unresolvable inflammation after a mild perturbation. Similar observations have been reported in lung where cytokine profile and infiltration of inflammatory cells show no difference between Ace2^{-/-} and WT mice until stimulation^{18, 19, 20}. In contrast, ACE2 deficiency led to increases in inflammation in mouse kidney and aorta even without perturbation^{21, 22}. Although the lung and cornea are disparate tissues, the similarity in inflammatory responses raises the interesting question of what are the underlying mechanisms driving inflammation.

As noted earlier in ACE2-deficient mice, cloudy corneas were frequently observed in older (>60 weeks) mice and sometimes only in one eye. This most likely reflects an earlier external trauma. However, it is also possible that cloudy corneas seen in older mice are the result of an enhanced accumulation of reactive oxygen species due to alterations in RAS, as it has been shown that ACE2-deficient mice exhibit increased oxidative stress²³. It is well established that with age, the well-developed corneal antioxidant defense systems diminish, leading to ROS accumulation and oxidative stress (²⁴ and references therein). This combination of ROS and oxidative stress can lead to many age-related ocular diseases of the anterior segment (for reviews see^{24, 25}). In other tissues, such as the brain, AngII has been shown to induce NADPH oxidase-dependent ROS production in isolated cerebral microvessels, resulting in cerebrovascular dysregulation²⁶.

In the ACE2-deficient mice, once an inflammatory response is initiated, inflammation persists becoming permanent, which remodels the stromal microenvironment. This microenvironmental change results in a wide range of epithelial phenotypes. The most dramatic is a cell fate switch from the transparent corneal epithelium to a keratinized, stratified squamous, psoriasiform-like epidermis. A similar cell-fate switch was reported in mice deficient in leucine-rich repeats and immunoglobulin-like domains 1 (LRIG1)²⁷. In these mice, loss of LRIG1 resulted in a proinflammatory state in the cornea through upregulation of Stat3. The persistence of the inflammation eventually resulted in the generation of an epithelium that resembled human psoriasis.

When ACE2 is silenced in a corneal epithelial cell line, interleukins (IL1A, IL1B, IL6), chemokines (CCL2, CXCL8), and TNFA, are all significantly elevated, resulting in a cytokine storm-like phenotype (²⁸ and references therein). We observed that losartan treatment significantly blocked the induction of ILIA, TNFA, and CCL2 by ACE2 depletion in cell cultures but only slightly altered the expression of IL1B and CXCL8, indicating a partial rescue. Losartan is one of the AngII receptor blockers (ARBs). Unlike other ARBs, the effect of losartan can be overcome by high concentrations of AngII, which means that losartan is surmountable²⁹. In addition, losartan needs to be converted in the liver to form its active metabolite EXP3174, which is 10-40 times more potent than losartan³⁰. These two features of losartan may explain why we only see partial rescue in vitro. Nonetheless, these findings clearly establish that the recruitment and activation of the macrophage component of the inflammatory response is related to AngII acting via the AT1 receptor.

In the aggregate, our findings suggests that provision of ACE2 may provide a new strategy for treating corneal inflammatory conditions as suggested, as well as other indications such as kidney disease.

Materials and Methods

Animal Studies:

Animal procedures were approved by the Northwestern University Animal Care and Use Committee. ACE2-deficient mice (Ace2^{-/-}) were developed on a C57B1/6 background³⁶. To generate debridement wounds in the central corneal epithelium, a rotating diamond burr was gently applied to the surface of the central cornea to remove the corneal epithelium while the peripheral corneal and limbal epithelia remained intact. Clinic images of mouse eyes were taken using a Leica dissecting scope. Wound closure was examined by topically applying 20 μL of 0.5% fluorescein in PBS and imaging the wound using a Leica dissecting scope under cobalt blue illumination. At the termination of the experiment, mouse whole eyes were fixed in 10% buffered formalin solution and paraffin embedded for histological analysis. H&E staining was conducted as described previously³⁷ and slides were imaged using an AxioVision Z1 fluorescence microscope system (Carl Zeiss, Oberkochen, Germany).

Immunohistochemistry of Human and Mouse Cornea

Normal human corneal tissues were obtained from the Eversight eye banks (Ann Arbor, Mich., USA) and embedded in paraffin blocks. Immunohistochemical (IHC) staining of human and Ace2^{-/-} mouse eyes were conducted as described previously³⁸. Antigen retrieval of the paraffin embedded sections was performed at 96° C. in pH 6.0 citrate buffer for 30 min. After blocking in 2.5% normal horse serum, sections were incubated overnight at 4° C. with antibodies recognizing: AngiotensinII (AngII) rabbit polyclonal antibody (Penlabs, San Carlos, Calif.), ACE2 goat polyclonal antibody (R&D systems, Minneapolis, Minn.), CD68 rabbit polyclonal antibody (Proteintech, Chicago, Ill.), CD3 rabbit polyclonal antibody (Proteintech, Chicago, Ill.), and CD11c rabbit polyclonal antibody (Proteintech, Chicago, Ill.) at 1:100 dilution. After rinsing with PBS containing 0.1% Tween (PBST), the samples were incubated with ImmPRESS-AP (alkaline phosphatase) Polymer Anti-Rabbit or Anti-Goat IgG Reagent (Vector lab, Burlingame, Calif.) at room temperature for 30 min. After rinsing with PBST, chromagen was detected using a Vector Red Alkaline Phosphatase Substrate Kit (Vector lab, Burlingame, Calif.) for 10-30 min. Samples were counterstained with hematoxylin, and dehydrated with graded ethanol and xylene. Images were taken using an AxioVision Z1 fluorescence microscope system (Carl Zeiss, Oberkochen, Germany).

Cell Culture

Immortalized corneal epithelial cells, hTCEpi, were cultured in keratinocyte serum free media (Thermo Fisher Scientific, Waltham, Mass., USA) as described before³⁹. For siRNA knock-down experiments, cells were transfected with 10 nM siRNA SMARTpools against ACE2 and non-target control (GE Dharmacon, Colorado, USA) as previously described³⁷. Two days after transfection, cells were treated with losartan (100 μM) for 24 hours, and processed for total RNA isolation.

Real Time Quantitative PCR (qPCR)

Total RNAs were isolated from hTCEpi cells and purified with a miRNeasy kit (Qiagen, Hilden, Germany). Real-time qPCR (RT-qPCR) was performed with a Roche LightCycler 96 System using the Roche FastStart Essential DNA Green Master (Roche, Branchburg, N.J., USA) according to the manufacturer's instructions.

Statistical Analysis

Unpaired t-test were performed to determine statistical significance. The data are shown as means±standard deviation (SD). The differences were considered significant for p values of <0.05. All experiments were replicated at least three times.

### Example 2 Prophetic: ACE2 Treatment Will Improve Diseased or Damaged Corneal Tissue

In this Example, young Ace2^{-/-} mice with bilateral clear corneas, and age-matched wild-type littermates will be used to test different ophthalmic compositions comprising an inactive vehicle and either: [1] ACE2 polypeptide (SEQ ID NO: 1); [2] ACE2 fragment (SEQ ID NO: 2); [3] ACE2 fragment (SEQ ID NO:3); [4] ACE2 fragment (SEQ ID NO:3) and ACE2 fragment (SEQ ID NO; 4) (or optionally SEQ ID NO:10 or SEQ ID NO:18). The control composition [5] will include vehicle only. The test compositions will be a preservative-free 10 mg/ml aqueous solution of the ACE2 peptide or variant, with 10 mM histidine HCl, 10% α,α-trehalose dihydrate, 0.01% polysorbate 20, pH 5.5. The test and control eye drops (10 μl) will be applied to the right eye of the mice according to three regimens of application: (a) every 2 h for 12 h daily, (b) four times daily, and (c) twice daily. Five mice will be included in each subgroup.

Small 1 mm circular debridement wounds will be made in both corneas of each mouse as described above in Example 1. Immediately following wounding, all mice will receive a topical application of one of compounds (1)-(5) and a topical application of a 0.5% fluorescein stain. The rate of epithelial healing will be visualized for one week as described in Example 1.

Anticipated results are presented in Table 1 and Table 2 below. Table 1 shows anticipated results in the ACE2^{-/-} mice. Table 2 shows anticipated results in the wild-type mice. Symbol + indicates observed improvement in healing as compared to the untreated controls in group 5. As is shown in Tables 1 and 2, it is anticipated that ACE2, either full-length or truncated, will improve corneal wound healing across all regimens tested. With respect to the wild-type mice, while natural healing is anticipated to be observed by 24 hours, it is also anticipated that the treatment groups will show enhanced (faster) healing than the untreated controls.

TABLE 1

| | Group 1 | Group 2 | Group 3 | Group 4 | Group 5 |
|---|---|---|---|---|---|
| | Regimen | Regimen | Regimen | Regimen | Regimen |
| ACE2^{-/-} | (a)/(b)/(c) | (a)/(b)/(c) | (a)/(b)/(c) | (a)/(b)/(c) | (a)/(b)/(c) |
| 24-hours | +/+/+ | +/+/+ | +/+/+ | +/+/+ | −/−/− |
| 3 days | +/+/+ | +/+/+ | +/+/+ | +/+/+ | −/−/− |
| 7 days | ++/++/++ | ++/++/++ | ++/++/++ | ++/++/++ | −/−/− |

TABLE 2

| | Group 1 | Group 2 | Group 3 | Group 4 | Group 5 |
|---|---|---|---|---|---|
| | Regimen | Regimen | Regimen | Regimen | Regimen |
| Wild-type | (a)/(b)/(c) | (a)/(b)/(c) | (a)/(b)/(c) | (a)/(b)/(c) | (a)/(b)/(c) |
| 24-hours | +/+/+ | +/+/+ | +/+/+ | +/+/+ | −/−/− |
| 3 days | ++/++/++ | ++/++/++ | ++/++/++ | ++/++/++ | +/+/+ |
| 7 days | ++/++/++ | ++/++/++ | ++/++/++ | ++/++/++ | ++/++/++ |

### BIBLIOGRAPHY AND REFERENCES CITED

1. Guy J L, Jackson R M, Acharya K R, Sturrock E D, Hooper N M, Turner A J. Angiotensin-converting enzyme-2 (ACE2): comparative modeling of the active site, specificity requirements, and chloride dependence. Biochemistry 42, 13185-13192 (2003).
2. Serfozo P, et al. Ang II (Angiotensin II) Conversion to Angiotensin-(1-7) in the Circulation Is POP (Prolyloligopeptidase)—Dependent and ACE2 (Angiotensin-Converting Enzyme 2)—Independent. Hypertension 75, 173-182 (2020).
3. Wysocki J, et al. Targeting the degradation of angiotensin II with recombinant angiotensin-converting enzyme 2: prevention of angiotensin II-dependent hypertension. Hypertension 55, 90-98 (2010).
4. Suzuki Y, Ruiz-Ortega M, Egido J. Angiotensin II: A double-edged sword in inflammation. J Nephrol 13, S101-S110 (2000).
5. Donoghue M, et al. A novel angiotensin-converting enzyme-related carboxypeptidase (ACE2) converts angiotensin Ito angiotensin 1-9. Circ Res 87, E1-9 (2000).
6. Tipnis S R, Hooper N M, Hyde R, Karran E, Christie G, Turner A J. A human homolog of angiotensin-converting enzyme. Cloning and functional expression as a captopril-insensitive carboxypeptidase. J Biol Chem 275, 33238-33243 (2000).
7. Tikellis C, et al. Identification of angiotensin converting enzyme 2 in the rodent retina. Curr Eye Res 29, 419-427 (2004).
8. Wysocki J, Schulze A, Batlle D. Novel Variants of Angiotensin Converting Enzyme-2 of Shorter Molecular Size to Target the Kidney Renin Angiotensin System. Biomolecules 9, (2019).
9. Lai C C, Shih T P, Ko W C, Tang H J, Hsueh P R. Severe acute respiratory syndrome coronavirus 2 (SARS-CoV-2) and coronavirus disease-2019 (COVID-19): The epidemic and the challenges. Int J Antimicrob Agents 55, 105924 (2020).
10. Stepp M A, et al. Wounding the cornea to learn how it heals. Experimental eye research 121, 178-193 (2014).
11. Gupta N, Kalaivani M, Tandon R. Comparison of prognostic value of Roper Hall and Dua classification systems in acute ocular burns. Br J Ophthalmol 95, 194-198 (2011).
12. Wilson S E, Mohan R R, Mohan R R, Ambrosio R, Jr., Hong J, Lee J. The corneal wound healing response: cytokine-mediated interaction of the epithelium, stroma, and inflammatory cells. Prog Retin Eye Res 20, 625-637 (2001).
13. Kusnadi A, et al. The Cytokine TNF Promotes Transcription Factor SREBP Activity and Binding to Inflammatory Genes to Activate Macrophages and Limit Tissue Repair. Immunity 51, 241-257 e249 (2019).
14. Park S H, et al. Type I interferons and the cytokine TNF cooperatively reprogram the macrophage epigenome to promote inflammatory activation. Nat Immunol 18, 1104-1116 (2017).
15. Zou K, Wang Y, Hu Y, Zheng L, Xu W, Li G. Specific tumor-derived CCL2 mediated by pyruvate kinase M2 in colorectal cancer cells contributes to macrophage recruitment in tumor microenvironment. Tumour Biol 39, 1010428317695962 (2017).
16. Chen C, et al. LNMAT1 promotes lymphatic metastasis of bladder cancer via CCL2 dependent macrophage recruitment. Nat Commun 9, 3826 (2018).
17. Lgic R. Four decdes of ocular renin-angiotensin and kallikrein-kinin systems (1977-2017). Experimental eye research 166, 74-83 (2018).
18. Imai Y, et al. Angiotensin-converting enzyme 2 protects from severe acute lung failure. Nature 436, 112-116 (2005).
19. Lin C I, et al. Instillation of particulate matter 2.5 induced acute lung injury and attenuated the injury recovery in ACE2 knockout mice. Int J Biol Sci 14, 253-265 (2018).
20. Kuba K, et al. A crucial role of angiotensin converting enzyme 2 (ACE2) in SARS coronavirus-induced lung injury. Nat Med 11, 875-879 (2005).
21. Jin H Y, et al. Deletion of angiotensin-converting enzyme 2 exacerbates renal inflammation and injury in apolipoprotein E-deficient mice through modulation of the nephrin and TNF-alpha-TNFRSF1A signaling. J Transl Med 13, 255 (2015).
22. Thomas M C, et al. Genetic Ace2 deficiency accentuates vascular inflammation and atherosclerosis in the ApoE knockout mouse. Circ Res 107, 888-897 (2010).
23. Wysocki J, et al. ACE2 deficiency increases NADPH-mediated oxidative stress in the kidney. Physiological reports 2, e00264 (2014).
24. Nita M, Grzybowski A. The Role of the Reactive Oxygen Species and Oxidative Stress in the Pathomechanism of the Age-Related Ocular Diseases and Other Pathologies of the Anterior and Posterior Eye Segments in Adults. Oxid Med Cell Longev 2016, U.S. Pat. No. 3,164,734 (2016).
25. Vallabh N A, Romano V, Willoughby C E. Mitochondrial dysfunction and oxidative stress in corneal disease. Mitochondrion 36, 103-113 (2017).
26. Kazama K, et al. Angiotensin II impairs neurovascular coupling in neocortex through NADPH oxidase-derived radicals. Circ Res 95, 1019-1026 (2004).
27. Nakamura T, et al. LRIG1 inhibits STAT3-dependent inflammation to maintain corneal homeostasis. J Clin Invest 124, 385-397 (2014).
28. Tisoncik J R, Korth M J, Simmons C P, Farrar J, Martin T R, Katze M G. Into the eye of the cytokine storm. Microbiol Mol Biol Rev 76, 16-32 (2012).
29. Burnier M. Angiotensin II type 1 receptor blockers. Circulation 103, 904-912 (2001).
30. Israili Z H. Clinical pharmacokinetics of angiotensin II (AT1) receptor blockers in hypertension. J Hum Hypertens 14 Suppl 1, S73-86 (2000).
31. Hanff T C, Harhay M O, Brown T S, Cohen J B, Mohareb A M. Is There an Association Between COVID-19 Mortality and the Renin-Angiotensin System-a Call for Epidemiologic Investigations. Clin Infect Dis, (2020).
32. Gralinski L E, Baric R S. Molecular pathology of emerging coronavirus infections. J Pathol 235, 185-195 (2015).
33. Zhou P, et al. A pneumonia outbreak associated with a new coronavirus of probable bat origin. Nature 579, 270-273 (2020).
34. Batlle D, Wysocki J, Satchell K. Soluble angiotensin-converting enzyme 2: a potential approach for coronavirus infection therapy? Clin Sci (Lond) 134, 543-545 (2020).
35. Xu Z, et al. Pathological findings of COVID-19 associated with acute respiratory distress syndrome. Lancet Respir Med, (2020).
36. Gurley S B, et al. Altered blood pressure responses and normal cardiac phenotype in ACE2-null mice. J Clin Invest 116, 2218-2225 (2006).
37. Kaplan N, et al. FIH-1 engages novel binding partners to positively influence epithelial proliferation via p63. FASEB J 34, 525-539 (2020).
38. Park J K, et al. MicroRNAs-103/107 coordinately regulate macropinocytosis and autophagy. The Journal of cell biology 215, 667-685 (2016).
39. Peng H, et al. microRNA-31/factor-inhibiting hypoxia-inducible factor 1 nexus regulates keratinocyte differentiation. Proceedings of the National Academy of Sciences of the United States of America 109, 14030-14034 (2012).

### Example 3: ACE2 Activity in Cornea of ACE2-Deficient Mice

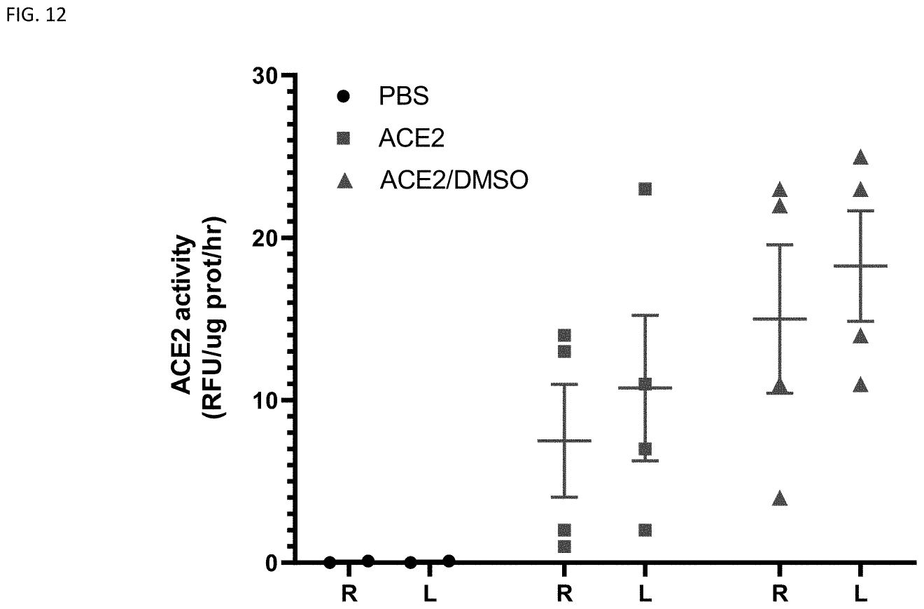

ACE2 activity was assessed in eyes isolated from ACE2-deficient mice. Human recombinant ACE2 1-618ABD dissolved in PBS (5 μg/μl) or dissolved in 0.5% DMSO in PBS ((5 μg/μ1) was applied topically to the cornea of ACE2-knockout (KO) mice. After one hour, the ACE2 activity in eyes of treated mice was compared to untreated mice which had been administered PBS. Detectable ACE2 activity was observed in mice administered ACE2 1-618ABD. DMSO appeared to enhance detectable ACE2 activity. (See FIG. 12).

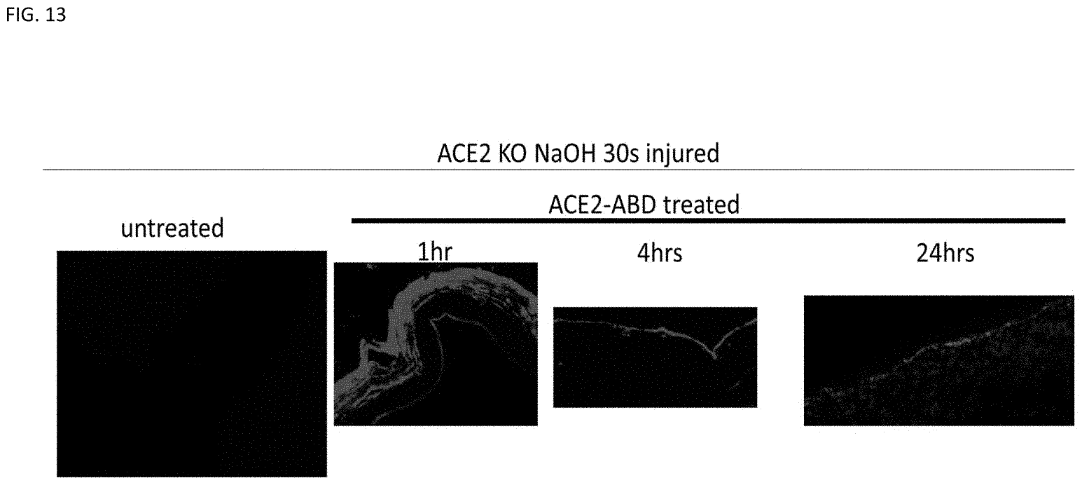

The corneas of ACE2-KO mice were treated with NaOH for 30 s and then were topically administered ACE2 1-618-ABD. The cornea were subjected to immunofluorescence using anti-ACE2 antibodies. (See FIG. 13). FIG. 13 provides the appearance of ACE2 staining by immunofluorescence in the cornea of ACE2 deficient mice after administration of ACE2 1-618ABD topically.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A method of treating a disease or condition of the cornea in a subject in need thereof, the method comprising: administering to the subject a composition comprising an ACE2 polypeptide or a variant thereof.

2. The method of claim 1, wherein the ACE2 polypeptide fragment or variant thereof has an ACE2 activity comprising cleaving AngII to Ang (1-7).

3. The method of claim 1, wherein the ACE2 polypeptide is selected from the group consisting of SEQ ID NO: 1, SEQ ID NO: 3, SEQ ID NO: 4, SEQ ID NO: 10 and SEQ ID NO: 18.

4. The method of claim 1, wherein the ACE2 polypeptide or the variant thereof is soluble in an aqueous solution.

5. The method of claim 1, wherein the ACE2 polypeptide or the variant thereof comprises a deletion which removes the transmembrane portion of the ACE2 polypeptide.

6. The method of claim 1, wherein the ACE2 polypeptide is a fusion polypeptide comprising (1) an ACE2 polypeptide selected from the group consisting of SEQ ID NO: 1, SEQ ID NO: 3, SEQ ID NO: 4, SEQ ID NO: 10, and SEQ ID NO: 12, and (2) a fusion partner selected from the group consisting of SEQ ID NO: 6, SEQ ID NO: 7, SEQ ID NO: 8, or SEQ ID NO: 9.

7. The method of claim 1, wherein the disease or condition comprises a traumatic injury to the cornea.

8. The method of claim 1, wherein the disease or condition comprises one or more symptoms selected from the group consisting of corneal deformation, corneal inflammation, corneal clouding, corneal neovascularization, and corneal edema.

9. The method of claim 8, wherein the symptom comprises inflammation.

10. The method of claim 1, wherein the disease or condition comprises one or more symptoms selected from the group consisting of increased expression of a marker of inflammation in the cornea, increased expression of ROS in the cornea, or increased expression of AngII in the cornea.

11. The method of claim 1, wherein the disease or condition is selected from the group consisting of: bullous keratopathy, pterygium, corneal ulcer, herpes simplex keratitis, herpes zoster ophthalmicus, herpes zoster keratitis, fungal keratitis, interstitial keratitis, keratoconjunctivitis sicca, keratomalacia, peripheral ulcerative keratitis, phlyctenular keratoconjunctivitis, superficial punctate keratitis, keratoconus, corneal dystrophies such as, but not limited to Fuch's Dystrophy, Lattice Dystrophy Type I, Lattice Dystrophy Type II, congenital stromal corneal dystrophy, Meesmann corneal dystrophy, and map dot fingerprint dystrophy, congenital corneal diseases such as, but not limited to complete LCAT deficiency, fish-eye disease, keratitis-ichthyosis-deafness syndrome, Peters anomaly, Peters plus syndrome; Bowen disease, Cogan syndrome, dry eye, dryness in the eye due to Sjogren syndrome, vitamin A deficiency, or LASIK eye surgery, infections, sensitivity to non-infectious bacteria or toxins, allergies, trachoma, river blindness, corneal transplant, recurrent corneal erosion, and tumors.

12. The method of claim 1, wherein administrating comprises extraocular delivery.

13. The method of claim 1, wherein administrating comprises intraocular delivery.

14. The method of claim 1, wherein administrating comprises topical delivery to the eye.

15. The method of claim 1, wherein administrating comprises topical delivery to the cornea.

16. The method of claim 1, wherein the composition comprises at least one additional active agent.

17. The method of claim 16, wherein the at least one additional active agent is selected from the group consisting of an anti-inflammatory agent, and an antibiotic.

18. The method of claim 1, wherein the ACE2 polypeptide comprises SEQ ID NO: 1.

19. The method of claim 1, wherein the ACE2 polypeptide comprises SEQ ID NO: 3.

20. The method of claim 1, wherein the ACE2 polypeptide comprises SEQ ID NO: 4, SEQ ID NO:10, or SEQ ID NO: 18.


SEQUENCE LISTING (16 sequences)

SEQ ID NO: 1 - protein, 805 aa, Homo sapiens [sequence omitted]

SEQ ID NO: 2 - protein, 805 aa, Mus musculus
MSSSSWLLLSLVAVTTAQSLTEENAKTFLNNFNQEAEDLSYQSSLASWNYNTNITEENAQKMSEAAAKWSAFYEEQSKTAQSFSLQEIQTPIIKRQLQALQQSGSSALSADKNKQLNTILNTMSTIYSTGKVCNPKNPQECLLLEPGLDEIMATSTDYNSRLWAWEGWRAEVGKQLRPLYEEYVVLKNEMARANNYNDYGDYWRGDYEAEGADGYNYNRNQLIEDVERTFAEIKPLYEHLHAYVRRKLMDTYPSYISPTGCLPAHLLGDMWGRFWTNLYPLTVPFAQKPNIDVTDAMMNQGWDAERIFQEAEKFFVSVGLPHMTQGFWANSMLTEPADGRKVVCHPTAWDLGHGDFRIKMCTKVTMDNFLTAHHEMGHIQYDMAYARQPFLLRNGANEGFHEAVGEIMSLSAATPKHLKSIGLLPSDFQEDSETEINFLLKQALTIVGTLPFTYMLEKWRWMVFRGEIPKEQWMKKWWEMKREIVGVVEPLPHDETYCDPASLFHVSNDYSFIRYYTRTIYQFQFQEALCQAAKYNGSLHKCDISNSTEAGQKLLKMLSLGNSEPWTKALENVVGARNMDVKPLLNYFQPLFDWLKEQNRNSFVGWNTEWSPYADQSIKVRISLKSALGANAYEWTNNEMFLFRSSVAYAMRKYFSIIKNQTVPFLEEDVRVSDLKPRVSFYFFVTSPQNVSDVIPRSEVEDAIRMSRGRINDVFGLNDNSLEFLGIHPTLEPPYQPPVTIWLIIFGVVMALVVVGIIILIVTGIKGRKKKNETKREENPYDSMDIGKGESNAGFQNSDDAQTSF

SEQ ID NO: 3 - protein, 619 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIK

SEQ ID NO: 4 - protein, 605 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVG

SEQ ID NO: 5 - protein, 46 aa, Artificial Sequence
  Variant of Streptococcal G-protein albumin binding domain III
LAEAKVLANRELDKYGVSDFYKRLINKAKTVEGVEALKLHILAALP

SEQ ID NO: 6 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 107 aa, Homo sapiens
GQPREPQVYTSPPSRDELTKNQVSLRCHVKGFYPSDIAVEWESNGQPENNYKTTKPVLDSDGSFFLYSKLTVDKSRWQQGNVFSCSVMHEALHNHYTQKSLSLSPGK

SEQ ID NO: 8 - protein, 107 aa, Artificial Sequence
  Modified amino acid sequence of the monomeric, disulfide-stabilized mCH3 fragment of human IgG
GQCREPQVYTSPPSRDELTKNQVSLRCHVKGFYPSDIAVEWESNGQPENNYKTTKPVLDSDGSFFLYSKLTVDKSRWQQGNVFSCSVMHECLHNHYTQKSLSLSPGK

SEQ ID NO: 9 - protein, 205 aa, Homo sapiens
LEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDCLSVFLNQLCVLHEKTPVSDRVTKCCTESLVNGRPCFSALEVDETYVPKEFNAETFTFHADICTLSEKERQIKKQTALVELVKHKPKATKEQLKAVMDDFAAFVEKCCKA

SEQ ID NO: 10 - protein, 618 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSI

SEQ ID NO: 11 - protein, 542 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKC

SEQ ID NO: 12 - protein, 522 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQ

SEQ ID NO: 13 - protein, 45 aa, Artificial Sequence
  Synthetic- Consensus Albumin Binding Domain (ABDCon)
LKEAKEKAIEELKKAGITSDYYFDLINKAKTVEGVNALKDEILKA

SEQ ID NO: 14 - protein, 18 aa, Artificial Sequence
  Synthetic- linker sequence
GGSSRSSSSGGGGSGGGG

SEQ ID NO: 15 - protein, 12 aa, Homo sapiens
EPKSCDKTHTCP

SEQ ID NO: 16 - protein, 13 aa, Artificial Sequence
  Synthetic- modified hinge region
KCHWECRGCRLVC